(19) United States
(12) Patent Application Publication
(10) Pub. No.: US 2023/0372374 A1
(43) Pub. Date: Nov. 23, 2023

(54) VETERINARY PHARMACEUTICAL FORMULATIONS

(71) Applicant: INCREVET, INC., Boston, MA (US)

(72) Inventors: Ning LI, Shanghai (CN); Michael J. HADD, San Jose, CA (US); Brian SEED, Boston, MA (US); Gopinath DEVARAJ, Telangana State (IN); Esther SEE, Auckland (NZ); Su WIN, Auckland (NZ)

(21) Appl. No.: 18/318,133

(22) Filed: May 16, 2023

(30) Foreign Application Priority Data
May 17, 2022 (WO) PCT/CN2022/093377

(51) Int. Cl. A61K 31/7034; A61K 9/20; A61K 47/26; A61K 47/10

(52) U.S. Cl. CPC A61K 31/7034; A61K 9/2068; A61K 9/2018; A61K 9/2054; A61K 9/2059; A61K 9/2009; A61K 9/2013; A61K 47/26; A61K 47/10

(57) ABSTRACT

Provided herein are oral immediate release formulations of bexagliflozin for administration to companion animals.

### CROSS-REFERENCES TO RELATED APPLICATIONS

This application claims priority to International Application No. PCT/CN2022/093377, filed May 17, 2022, which is incorporated herein in its entirety by reference for all purposes.

### TECHNICAL FIELD

The invention provides pharmaceutical formulations of bexagliflozin useful for treating diabetes mellitus, hypertension, renal failure, or cardiac failure in companion animals. Oral formulations of bexagliflozin that are convenient to administer, are readily accepted by cats and dogs, and that have improved pharmacokinetic properties, are described.

### BACKGROUND OF THE INVENTION

Bexagliflozin is a C-aryl glucoside inhibitor of human Sodium Glucose Linked Transporter 2 (SGLT2), the renal reuptake transporter responsible for the recovery of the majority of glucose eliminated from plasma in the renal glomerular filtrate. In recent years inhibitors of SGLT2 (and to some extent, the related transporter SGLT1) have been found to have beneficial effects in humans for the treatment of diabetes, kidney disease and congestive heart failure.

Of the potential uses for SGLT inhibitors for diseases of companion animals, one of the opportunities most relevant to this invention is for improvement in the management of feline diabetes mellitus. At present, there are no approved oral hypoglycemic agents for managing diabetes in cats. The standard of care for feline diabetes requires twice daily injections of insulin, titrated to effect. Cats show substantial inter-individual variation in insulin sensitivity and must be carefully observed to ensure that a fatal or neurologically catastrophic hypoglycemia does not occur. Although the administration of insulin can help control diabetes and slow disease progression, providing the proper dose and timing of insulin can be challenging. For example, it is recommended that administration of insulin be timed around a meal; however, consistent timing of insulin with meals can be difficult for cat owners to achieve.

As such, one need in the art is for more effective methods and compositions for managing feline diabetes and pre-diabetes. Bexagliflozin has promising attributes for this indication. Inhibition of SGLT2 by bexagliflozin results in a prominent and saturable glucosuria in mice, rats, cats, dogs, rabbits, monkeys and humans.

Bexagliflozin has been studied in diabetic mice, rats, cats and humans. In each case the compound has been found to reduce blood glucose levels and improve long-term measures of glycemic control, such as HbA_{1c} in rodents or humans, or fructosamine in cats. Experiments using rodent genetic models of diabetes have shown that even in the presence of a florid pre-existing glucosuria, bexagliflozin can produce a reduction in blood glucose levels and a partial alleviation of disease. Hence, the existence of glucosuria does not preclude the application of bexagliflozin for the treatment of diabetes.

Bexagliflozin has also been found to have a favorable effect on the fluid retention caused by the thiazolidinedione anti-diabetic agent pioglitazone (US20190343853A1). This effect is presumably at least in part attributable to the natriuresis induced by bexagliflozin, which may be a consequence of osmotic diuresis as well as a perturbation in sodium balance due to blockade of sodium cotransport from the renal filtrate. Through its effects on kidneys, bexagliflozin can exert a beneficial action on chronic kidney disease and heart failure in dogs and cats.

In cats, the action of bexagliflozin is unusually potent and qualitatively superior to that observed in other organisms. In a high fraction of diabetic cats, bexagliflozin has produced a clinical remission of the disease, resulting in serum fructosamine concentrations that fall within the reference range for healthy cats (U.S. Ser. No. 16/818,026). Despite the high potency of bexagliflozin in cats, signs or objective measurements of clinically significant hypoglycemia have not been observed to date. The combination of high potency and low risk make bexagliflozin a superior choice for the treatment of feline diabetes. Of particular interest and utility is the observation that bexagliflozin as a monotherapy has proven to restore fructosamine levels to within the testing laboratory normal reference range in a majority of cats in a field study in which the cats were administered their medications by owners in an unsupervised (home) context (U.S. Ser. No. 16/818,026).

Bexagliflozin (also named EGT0001442, EGT1442, THR1442, THR0001442) has been found to be useful for the treatment and management of various conditions, including human diabetes (see: Zhang et al. (2011) Pharmacol Res 63(4):284-93; Allegretti et al. (2019) Am J Kidney Dis. 74:328 doi: 10.1053/j.ajkd.2019.03.417; Zhang et al. (2019) Xenobiotica doi: 10.1080/00498254.2019.1654634). It has been shown to be well-tolerated and to provide a durable, clinically meaningful improvement in glycemic control in humans, as well as a reduction in body mass and blood pressure in diabetic adults (Halvorsen et al. (2019) Diabetes Obes Metab doi: 10.1111/dom.13833, Halvorsen et al. (2019) Diabetes Obes Metab 21:2248 doi: 10.1111/dom.13801).

Bexagliflozin has also been reported to be useful as an adjunct to insulin for the management of diabetes in cats, producing improvements in glycemic control and reduction in insulin dosage (Benedict et al. (2022) Can J Vet Res 86:52-58).

Bexagliflozin has been found to decrease the rate of reduction of glomerular filtration rate in humans, thereby impeding the progress of kidney disease, and has been found to produce a hazard ratio for the reduction in major adverse cardiovascular events of 0.774, indicating a low potential for cardiovascular risk.

Delivery of bexagliflozin to cats in need of treatment for diabetes can be achieved with oral dosing. The desired features for oral dosing include ease of dispensing, stability to normal storage conditions, promotion of absorption of the drug to be delivered, and ready acceptance by the animal to be treated. For the last of these features, administration of oral dosage forms to cats poses particular challenges, as cats are well-known to be difficult to dose with conventional solid dosage forms. As a result, medications for cats are often prepared as oral solutions.

For a chronic disease such as diabetes, the importance of a compatible dosage form is magnified, as cats will associate specific environmental cues with an imminent exposure to an undesired product and take evasive action, such as hiding, showing escape behaviors, or exhibiting active aggressive reactions upon initiation of dosing. Thus the acceptability of a formulation is a highly important design element for a medication intended to be administered on a daily basis.

A preferred dosing system places few requirements on the owner. For example, required preparations, such as to provide reconstitution of solutions, or precise measurement of volumes for delivery by a dropper or syringe dispenser, are less attractive than dosing systems that allow delivery of a single preformulated unit to accomplish the therapeutic objective. In human disease this is often accomplished by providing single tablet dosages that provide the treatment agent in a convenient, once-daily administration regimen. However, for delivery to cats, this has been difficult to achieve in many cases.

### SUMMARY

In some aspects, provided herein are formulations comprising bexagliflozin for administration to a companion animal. In some embodiments, the formulation is a tablet, a capsule, a softgel, or a liquid formulation. In some embodiments said formulation is a tablet.

In some aspects, provided herein are immediate release tablet formulations comprising bexagliflozin for administration to a companion animal.

In some embodiments, the immediate release formation in an in vitro dissolution test releases at least 70% of its bexagliflozin after 10 minutes. In some embodiments, the immediate release formation in an in vitro dissolution test releases at least 85% of its bexagliflozin after 30 minutes in a solution of 0.1N HCl at 37±0.5° C. in a USP Apparatus 2 (a paddle apparatus) with a paddle speed of about 75 rpm. In some embodiments, the immediate release formation releases of >41.2% of the bexagliflozin dosage after 5 minutes and ≥80% of the bexagliflozin after 30 minutes. In some embodiments, the immediate release formation in an in vitro dissolution test releases 5.4-78.4% of the bexagliflozin after 10 minutes and/or between 80.1-86.2% of the bexagliflozin after 15 minutes.

In some embodiments, the formulations of the current disclosure include a palatant. In some embodiments, the palatant comprises tuna, salmon, cream, beef, peanut, catnip, chicken liver powder, poultry extract, avian hydrolyzed liver, butter, or bacon flavoring. In some embodiments, the palatant comprises a meat or liver flavor. In some embodiments, the palatant comprises hydrolyzed chicken liver.

In some embodiments, tablet formulations of the present disclosure can include at least one ingredient selected from one or more fillers, one or more glidants, one or more lubricants, and one or more binders, or other ingredients.

In some embodiments the companion animal is a cat. In some embodiments, the companion animal is a dog.

In some aspects, provided herein is a tablet containing 15 mg of bexagliflozin and producing an f₂ value ≥50 when compared to a reference tablet of the formulation of Table 35, when tested by dissolution in USP Apparatus 2 (paddles) containing 500 mL of 0.1 N HCl at 37° C.±0.5° C., stirred at 75 rpm.

In some aspects, provided herein is a tablet having the following composition per tablet: bexagliflozin, between 10-20 mg; lactose monohydrate, between 17.5-27.5 mg; microcrystalline cellulose, between 20.5-30.5 mg; palatant, between 7.5-11 mg, pregelatinized starch, between 3-5 mg, colloidal silicon dioxide, between 1.5-2.5 mg; and magnesium stearate, between 0.75-1.25 mg.

In some aspects, provided herein is a tablet containing 15 mg of bexagliflozin and producing an f₂ value ≥50 when compared to a reference tablet of the formulation of Table 38, when tested by dissolution in USP Apparatus 2 (paddles) containing 500 mL of 0.1 N HCl at 37° C.±0.5° C., stirred at 75 rpm.

In some aspects, provided herein is a method for treating a companion animal suffering from a disease or syndrome susceptible to treatment with an SGLT2 inhibitor, comprising a step of administering to the animal the any formulation described herein. In some embodiments, the companion animal is diagnosed with diabetes mellitus or pre-diabetes mellitus. In some embodiments, the companion animal is diagnosed with type 1 diabetes mellitus. In some embodiments, the companion animal is diagnosed with type 2 diabetes mellitus. In some embodiments, the companion animal is diagnosed with hypertension. In some embodiments, the companion animal is diagnosed with renal failure. In some embodiments, the companion animal is diagnosed with cardiac failure.

In some aspects, provided herein is a liquid formulation containing bexagliflozin, 30 mg/mL, ethanol, 125 to 175 mg/mL, glycerol, 250 to 300 mg/mL, PEG-400, 125 to 175 mg/mL, polysorbate 80, 25 to 75 mg/mL, one or more optional palatants, 1 to 15 mg/mL and with the remainder comprising an aqueous buffer with pH between 6.0 and 8.0.

In some embodiments, the formulations comprising bexagliflozin described herein, after delivery to an appropriately constituted cohort of healthy adult cats in the fasted state, provides a mean plasma bexagliflozin AUC₀₋₂₄ greater than 1000 ng h mL⁻¹ per mg kg⁻¹ of bexagliflozin when tested at 3 mg kg⁻¹ of bexagliflozin. In some embodiments, this formulation is a tablet formulation.

In some embodiments, the formulations comprising bexagliflozin described herein, after delivery to an appropriately constituted cohort of healthy adult cats in the fasted state, provides a mean plasma bexagliflozin Cₘₐₓ greater than 300 ng mL⁻¹ per mg kg⁻¹ of bexagliflozin when tested at 3 mg kg⁻¹ of bexagliflozin. In some embodiments, this formulation is a tablet formulation.

In some aspects, provided herein is a batch of bexagliflozin veterinary tablets that include 15 mg of bexagliflozin wherein, upon administration to an appropriately constituted cohort of healthy fasted subjects, a first representative sample set of tablets from the batch provides on one occasion a first mean logarithm of the Cₘₐₓ and a first mean logarithm of the AUC₀₋ₜ, and a second representative sample of tablets from the batch produces on a different occasion a second mean logarithm of the Cₘₐₓ and a second mean logarithm of the AUC₀₋ₜ, and wherein the differences between the first and second mean logarithms of the Cₘₐₓ and between the first and second mean logarithms of the AUC₀₋ₜ both exhibit 90% confidence intervals, the endpoints of which lie between −0.51083 and +0.51083.

In some aspects, provided herein is a batch of bexagliflozin veterinary tablets that include 15 mg of bexagliflozin wherein, upon administration to an appropriately constituted cohort of healthy fasted subjects, a first representative sample set of tablets from the batch provides on one occasion a first mean logarithm of the Cₘₐₓ and a first mean logarithm of the AUC₀₋ₜ, and a second representative sample of tablets from the batch produces on a different occasion a second mean logarithm of the Cₘₐₓ and a second mean logarithm of the AUC₀₋ₜ, and wherein the differences between the first and second mean logarithms of the Cₘₐₓ and between the first and second mean logarithms of the AUC₀₋ₜ both exhibit 90% confidence intervals, the endpoints of which lie between −0.28768 and +0.28768.

In some aspects, provided herein is a batch of bexagliflozin veterinary tablets that include 15 mg of bexagliflozin wherein, upon administration to an appropriately constituted cohort of healthy fasted subjects, a first representative sample set of tablets from the batch provides on one occasion a first mean logarithm of the Cₘₐₓ and a first mean logarithm of the AUC₀₋ₜ, and a second representative sample of tablets from the batch produces on a different occasion a second mean logarithm of the Cₘₐₓ and a second mean logarithm of the AUC₀₋ₜ, and wherein the differences between the first and second mean logarithms of the Cₘₐₓ and between the first and second mean logarithms of the AUC₀₋ₜ both exhibit 90% confidence intervals, the endpoints of which lie between −0.22314 and +0.22314.

In some aspects, provided herein is a batch of bexagliflozin veterinary tablets that include 15 mg of bexagliflozin wherein, upon administration to an appropriately constituted cohort of healthy fasted subjects, a representative sample set of tablets from the batch provides a first mean logarithm of the Cₘₐₓ and a first mean logarithm of the AUC₀₋ₜ, and a representative sample of tablets from a reference batch of 15 mg bexagliflozin veterinary tablets produces a second mean logarithm of the Cₘₐₓ and a second mean logarithm of the AUC₀₋ₜ, and wherein the differences between the first and second mean logarithms of the Cₘₐₓ and between the first and second mean logarithms of the AUC₀₋ₜ both exhibit 90% confidence intervals, the endpoints of which lie between −0.51083 and +0.51083.

In some aspects, provided herein is a batch of bexagliflozin veterinary tablets that include 15 mg of bexagliflozin wherein, upon administration to an appropriately constituted cohort of healthy fasted subjects, a representative sample set of tablets from the batch provides a first mean logarithm of the Cₘₐₓ and a first mean logarithm of the AUC₀₋ₜ, and a representative sample of tablets from a reference batch of 15 mg bexagliflozin veterinary tablets produces a second mean logarithm of the Cₘₐₓ and a second mean logarithm of the AUC₀₋ₜ, and wherein the differences between the first and second mean logarithms of the Cₘₐₓ and between the first and second mean logarithms of the AUC₀₋ₜ both exhibit 90% confidence intervals, the endpoints of which lie between −0.28768 and +0.28768.

In some aspects, provided herein is a batch of bexagliflozin veterinary tablets that include 15 mg of bexagliflozin wherein, upon administration to an appropriately constituted cohort of healthy fasted subjects, a representative sample set of tablets from the batch provides a first mean logarithm of the Cₘₐₓ and a first mean logarithm of the AUC₀₋ₜ, and a representative sample of tablets from a reference batch of 15 mg bexagliflozin veterinary tablets produces a second mean logarithm of the Cₘₐₓ and a second mean logarithm of the AUC₀₋ₜ, and wherein the differences between the first and second mean logarithms of the Cₘₐₓ and between the first and second mean logarithms of the AUC₀₋ₜ both exhibit 90% confidence intervals, the endpoints of which lie between −0.22314 and +0.22314.

A kit comprising a formulation described herein and instructions for use. In some embodiments, the kit also includes another therapeutic agent.

### BRIEF DESCRIPTION OF THE DRAWINGS

FIG. 1 shows the bexagliflozin plasma concentration following capsule dosing.

FIG. 2 shows the AUC₀₋₂₄ as a function of dose following capsule dosing.

FIG. 3 shows the comparison of 2 liquid formulations and capsules at 3 mg kg⁻¹.

FIG. 4 shows repeat dosing of DEGEE/PEG-400 liquid formulations.

FIG. 5 shows dosing from formulations containing PEG-400 and other solvents.

FIG. 6 shows bexagliflozin plasma concentration after softgel dosing.

FIG. 7 shows plasma concentration following tablet dosing.

FIG. 8 shows bexagliflozin concentration as a function of time after dosing in the fasted state of the initial and three alternate formulations.

FIG. 9 shows bexagliflozin concentration as a function of time after dosing of the DB formulation in the fasted or fed states.

FIG. 10 shows bexagliflozin concentration as a function of time for 5.5 mm tablets containing bexagliflozin, 15 mg.

FIG. 11 shows urinary glucose excretion for a DEGEE liquid formulation as a function of dose.

FIG. 12 shows urinary glucose excretion for tablet and capsule formulations as a function of dose.

FIG. 13 shows impurity as a function of time at 40° C., 75% RH.

### DETAILED DESCRIPTION OF THE INVENTION

### I. General

The present disclosure provides oral formulations for administering bexagliflozin to companion animals. Companion animals, particularly felines, can be difficult to dose with pharmaceutical agents. The formulations disclosed herein provide not only desirable stability and pharmacological properties, but the formulations have a very low dosing rejection rate.

### II. Definitions

As used herein, the term “administering” means delivering by oral, buccal, nasal, rectal, vaginal or cutaneous routes or other topical contact, or by intravenous, intraperitoneal, intramuscular, intralesional or subcutaneous routes, or by the implantation of a slow-release device or preparation such as a pump, gel, reservoir or erodible substance to a subject. Administration can be by any route including parenteral, and transmucosal (e.g., oral, nasal, vaginal, rectal, or transdermal). Parenteral administration includes, e.g., intravenous, intramuscular, intra-arterial, intradermal, subcutaneous, intraperitoneal, intraventricular, intrathecal and intracranial. Other modes of delivery include, but are not limited to, the use of liposomal formulations, intravenous infusion, transdermal patches, and the like.

As used herein “antidiabetic agent” refers to a composition comprising medicines, medications, or medicaments generally used for the management of diabetes in animals. It is generally accepted that oral medications for the treatment of type 2 diabetes mellitus in humans have little utility for the management of feline diabetes. To date, no oral medication has been approved for the management of feline diabetes by a regulatory authority of the United States, the European Union, or Japan.

As used herein, the phrase “appropriately constituted cohort” refers to a collection of test subjects that typically consists of healthy individuals of both sexes in a sample size that provides appropriate power to estimate the desired pharmacokinetic parameter. A sample size that provides appropriate power can be calculated as described below. In routine practice, for example, to demonstrate bioequivalence for regulatory purposes, twelve or more subjects of each sex are often employed, or a total sample size of 24 subjects if sex is not balanced. Although it may not be a regulatory requirement, it is to be understood that for the purposes of determining whether or not a test article is a composition of the present invention, the experimental cohort should be constituted from individuals near the midpoint of the healthy population of young adults as a whole, so that, for example, the cohort would not contain a preponderance of individuals of high or low body mass, or exceptionally lean or obese body condition, or of elderly individuals or individuals with diseases or conditions that might inherently affect the absorption, distribution, metabolism or excretion of bexagliflozin.

As used herein, the term “AUC” means the estimated area under the curve of the plasma concentration of an analyte as a function of time. AUC is calculated here by the linear trapezoidal rule, following which the AUC between two timepoints is taken to be the average of the concentrations at the two timepoints, multiplied by the time between timepoints.

As used herein, the term “AUC₀₋₂₈” means the AUC from the time of dosing to the time 24 h following dosing.

As used herein, the term “AUC_{0-∞}” means the AUC from time 0 to infinity, as produced by extrapolation of a simple (monophasic) exponential decay. AUC_{0-∞}=AUC₀₋ₜ+Cₗₐₛₜ/kₑₗ, where Cₗₐₛₜ is the last quantifiable concentration expressed in the concentration units of the AUC and kₑₗ is the terminal elimination rate constant, expressed in the reciprocal of the time unit by which the AUC is expressed (typically hours).

As used herein, the term “AUC₀₋ₜ” means the AUC from the time of dosing to the time of the last quantifiable measurement.

As used herein, the term “batch” describes a collection of discrete dosage forms such as oral solid dosage forms or unit liquid dosage forms that can range in size from 100 units up to a complete manufacturing run (e.g., all of the units that are made from the same initial quantity of material and have undergone the same series of manufacturing operations, or any aggregate quantity of units that have undergone similar manufacturing operations and are pooled for testing or distribution purposes). The definition of “manufacturing batch” includes that provided by 21 USC 201.3, i.e., a “specific quantity of a drug or other material that is intended to have uniform character and quality, within specified limits, and is produced according to a single manufacturing order during the same cycle of manufacture.”

As used herein “bexagliflozin” refers to (2S,3R,4R,5S,6R)-2-(4-chloro-3-(4-(2-cyclopropoxyethoxy)benzyl)phenyl)-6-(hydroxymethyl)tetrahydro-2H-pyran-3,4,5-triol, having the chemical structure:

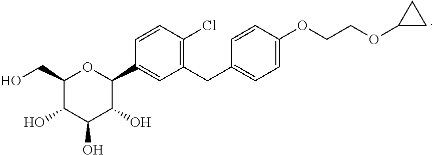 [SMILES: OC[C@H]1O[C@@H](c2ccc(Cl)c(Cc3ccc(OCCOC4CC4)cc3)c2)[C@H](O)[C@@H](O)[C@@H]1O]

As used herein, the word “cat” when used as an adjective and the word “feline” are used interchangeably and mean of or pertaining to an animal from the family Felidae, including particularly a member of that family that is maintained as a pet or companion animal and that typically belongs to the genus Felis, species Silvestris catus or species catus and is often referred to as a domestic cat or house cat.

As used herein, the word “cat” when used as a noun refers to a feline animal.

As used herein, the term “CL/F” means the means the apparent clearance, which is the clearance CL divided by the bioavailability F. CL/F is the measured clearance, whereas CL is an inferred quantity, except when the bioavailability is 100%.

As used herein, the term “Cₘₐₓ” means the maximum observed plasma concentration of an analyte.

As used herein, the term “Cₘᵢₙ” means the lowest observed plasma concentration, typically obtained as the value prior to a repeat dosing in a regularly scheduled dosing regimen. For example, for daily dosing Cₘᵢₙ is often recorded 24 hours after the previous dose.

The terms “d(0.1)”, “d(0.5)” and “d(0.9)” describe the threshold diameters for particles falling in the smallest 10%, 50% and 90% of the total volume of all particles. Thus at d(0.9), 90% of the volume of the sample can be found in particles of smaller diameter than d(0.9).

As used herein, the phrase “dosing failure” when applied to the circumstances surrounding the administration of a formulation indicates that the fraction of the formulation delivered to the animal had been less than 50% of the amount attempted to be delivered.

As used herein, the phrase “dosing rejection” when applied to the circumstances surrounding the administration of a formulation indicates that the animal to which the formulation had been attempted to be delivered had evaded or resisted dosing, for example, by fleeing, struggling, clawing at, biting, or attempting to injure the person administering the formulation, or exhibited post-dosing signs of distress, such as foaming, hypersalivation or vomiting.

As used herein, the word “fasted” when applied to the circumstances surrounding the collection of a specimen for testing indicates that the animal from which the specimen was drawn had been deprived of food for an extended period of time, typically 8 hours or longer and not unusually overnight if the specimen is taken in the morning. A fasted sample is useful for the measurements of formulations with a performance affected by feeding.

As used herein, the word “fed” when applied to the circumstances surrounding the collection of a specimen for testing indicates that the animal from which the specimen was drawn had been allowed to consume a standard meal within a short interval (not more than 30 minutes) of dose administration. Comparison of pharmacokinetics and pharmacodynamics following fasted and fed dosing can indicate if dosing instructions must stipulate a particular prandial state.

As used herein, “kₑₗ” means the first-order rate constant for elimination of a substance from plasma during the terminal phase of concentration decline. During this phase, if the kinetics are first-order, the concentration of the substance as a function of time, c(t), will be described by d c(t)/dt=−kₑₗ c(t). If the kinetics are not first-order, kₑₗ cannot be defined.

As used herein, the term “logarithm” refers by default to the natural logarithm, often written as a function of argument x as ln(x), where for avoidance of doubt, x=e^{ln(x)}. If the base of the logarithm is 10, the logarithm is referred to as the decimal logarithm, and written as a function of argument y as log₁₀(y), where, for avoidance of doubt, y=10^{log}^{₁₀}^{(y)}.

As used herein, “managing diabetes” or “management of diabetes” refers to the process by which an owner or other person responsible for the care of an animal addresses the disease by specific measures intended to palliate or cure the disease or provide relief of symptoms or change the perceived health of the animal by various means. Such means can include a change in the diet of the animal, including provision of a special or prescription diet or other change in the type or amount of food offered, or the encouragement or provision of activities that result in increased exertion or metabolic energy expenditure, or the provision of herbal preparations, dietary supplements or medicaments.

As used herein, the term “palatant” refers to an excipient that is added to a formulation to enhance the palatability of the formulation to a companion animal. A palatant is a flavoring agent that is tailored to the medicament and to the target animal species. Some palatants are specific to species, and others may comprise flavors or flavor combinations that are appealing to multiple species. Palatants often take human perceptions into account, as it has been found, for example, that compositions redolent of rotting flesh are attractive to dogs but repel humans, who tend not to provide their pets with products having odors they find objectionable.

As used herein, “plasma concentration” refers to an analyte concentration that is obtained by measurement of the liquid phase of whole, typically venous, blood that has been separated from the cellular components of the blood in such a manner that the blood does not coagulate.

As used herein, the term “representative”, when applied to a unit or sample of a batch, means a unit or sample that is not pre-selected for any particular character, such as weight, density, hardness or hue of coating, that is free from manufacturing defects and that is drawn substantially at random from the batch.

As used herein, the phrase “sample set” refers to a collection of units or samples that can be individually or collectively analyzed to estimate the properties of a batch or population as a whole. When used in connection with in vitro or in vivo testing of tablet properties, the sample set refers to a collection that is individually tested, and from which the properties of the batch of tablets as a whole are estimated.

The properties defined for any particular unit (e.g., a tablet, or defined fluid volume) are to be understood as being the properties of a representative unit drawn from a manufacturing batch, the members of which impart or exhibit the referenced properties in an appropriate test typically consuming multiple units from the manufacturing batch. Thus, when a unit is said to produce a particular pharmacokinetic parameter, it will be understood that this parameter will typically be measured after administration of representative specimens of a manufacturing batch of which that unit is an exemplar, and an appropriate statistical characterization of the results will be calculated.

For oral solid dosage forms, the unit of sampling is typically an individual dose, whereas for liquids the unit of sampling is often a bottle or similar reservoir containing the oral solution. From the reservoir is withdrawn or delivered the specified volume for testing, and the testing is intended to confirm that independently selected reservoirs contain contents of similar potency, palatability and composition. In some embodiments the reservoir is a single-use (single dose) delivery system, in which case the unit of sampling is the single-use article.

When a pharmacokinetic parameter is defined as having a certain range of values, it is to be understood that administration of representative specimens of a manufacturing batch of which that unit is an exemplar would produce, in an appropriately constituted experimental cohort, the characterized parameter (e.g., the mean or the median) falling within the stated range of values.

For instance, when tablets are said to produce a statistical measure (e.g., a mean Cₘₐₓ) falling within a certain range of values, it is to be understood that administration of representative specimens of a manufacturing batch of which that tablet is an exemplar would produce, in an appropriately constituted cohort, the statistical measure (e.g., the mean Cₘₐₓ) falling within the stated range of values.

Similarly, when liquid formulations produce a statistical measure falling within a certain range of values, it is to be understood that delivery of the test volume from representative reservoirs chosen from a manufacturing batch would produce, in an appropriately constituted cohort, the statistical measure (e.g., the mean Cₘₐₓ) falling within the stated range of values.

As used herein, the phrase “sample size that provides appropriate power” to estimate a pharmacokinetic parameter is the number of individuals in the cohort needed to achieve a discrimination of a particular degree between groups subjected to two experimental conditions, for example, having consumed a medication from one source or from another. Methods of calculating statistical power are well-known in the art. In its simplest form, statistical power describes the probability of obtaining a statistically significant result in a study in which the predicted difference actually exists between two populations. A power calculation is often cast as the determination of the minimum sample size to detect a true intergroup difference with a specified likelihood of failure due to randomness. For example, a 90% power means that in 9 out of 10 studies a statistically significant result will emerge, but in 1 out of 10, significance will not be achieved even though the difference is present. Hence, 100% minus the power is the probability of a false negative. Typical power values in testing pharmacokinetic parameters are 90% or greater and for definiteness “appropriate power” will be defined here as 95% or greater.

To perform a power calculation, the variability in the measure to be taken, usually expressed as a standard deviation, and the difference to be detected (the difference in the values of the measure from the two groups to be detected) must be input. If there is substantial uncertainty about the standard deviation of a measure in a population, it can be empirically determined. When used in the setting of noninferiority determinations, power calculations are used to estimate the sample size needed to confirm that the difference between two groups is less than a certain quantity. For example, bioequivalence studies are two-sided noninferiority tests that aim to demonstrate that the difference between two preparations falls within certain bounds.

When the prandial state, e.g., fasted or fed, is specified, the fasted state is to be achieved for each subject by withdrawal of food for at least eight hours prior to ingestion of a formulation and the fed state is to be achieved for each subject by consumption of a standard meal as regularly provided for the sustenance of the subject, with delivery of the formulation no later than 30 minutes after consumption of the meal.

As used herein, “serum concentration” refers to an analyte concentration that is measured in the liquid phase of whole, typically venous, blood that has been allowed to coagulate. Serum consists substantially of plasma depleted of coagulation factors and enriched for the contents of platelet granules.

As used herein, the phrase “solid oral dosage form” means any solid (or semi-solid) dosage form which can be administered orally. It can take the form of a tablet, a solid pill, a capsule, a caplet, a rigid or soft encapsulated gel or encapsulated liquid including a softgel, or combinations or concretions of such as may be present in layers or subcomponents such as beads, droplets or particles of various shapes and of differing properties embedded in a matrix or contained in a capsule or caplet.

As used herein, the phrase “substantially at random,” as it refers to the sampling of units of manufacture from a batch containing many such units, means either completely at random, such that every unit in the batch has an equal probability of being selected, or chosen by a process that aims to achieve a practical balanced representation of the batch being sampled. For example, representative units may be drawn at regular intervals during production to avoid a sampling imbalance in which units with slightly different properties, e.g., as produced from the beginning or end of a run, are overrepresented. Such units would be said to be drawn substantially at random from the batch.

As used herein, the term “t_{1/2}” means the terminal half-life, also referred to as the elimination half-life. If the empirically determined terminal elimination kinetics are not first-order in time, t_{1/2} cannot be defined. t_{1/2}=−ln(2)/kₑₗ≈0.693/kₑₗ.

As used herein, the term “Tₘₐₓ” means the time of observation of Cₘₐₓ.

As used herein, the term “V_{z}/F” means the means the apparent volume of distribution, which is the volume of distribution V_{z} divided by the bioavailability F.

The properties defined for any particular unit (e.g., a tablet) are to be understood as being the properties of a representative unit drawn from a manufacturing batch, the members of which impart or exhibit the referenced properties in an appropriate test typically consuming multiple units from the manufacturing batch. Thus, when a unit is said to produce a particular pharmacokinetic parameter, it will be understood that this parameter will typically be measured after administration of representative specimens of a manufacturing batch of which that unit is an exemplar, and an appropriate statistical characterization of the results will be calculated. Parameters based on plasma bexagliflozin concentrations (e.g., Cₘₐₓ and AUC) will typically be characterized as geometric means, whereas the Tₘₐₓ will typically be characterized by the population median. Furthermore, when a pharmacokinetic parameter is defined as having a certain range of values, it is to be understood that administration of representative specimens of a manufacturing batch of which that unit is an exemplar would produce, in an appropriately constituted experimental cohort, the characterized parameter (e.g., the geometric mean or the median) falling within the stated range of values.

For instance, when tablets are said to produce a statistical measure (e.g., a geometric mean Cₘₐₓ) falling within a certain range of values, it is to be understood that administration of representative specimens of a manufacturing batch of which that tablet is an exemplar would produce, in an appropriately constituted cohort, the statistical measure (e.g., the geometric mean Cₘₐₓ) falling within the stated range of values.

### III. Embodiments

Provided herein are oral formulations of bexagliflozin for administration to companion animals. In some embodiments, the formulation is a tablet, a capsule, a softgel, or a liquid formulation. Further details related to possible components for these formulations are provided in subsections A-E, below.

In some aspects, the formulations described herein are characterized by an in vitro dissolution test releases at least 70% of its bexagliflozin after 10 minutes and releases at least 85% of its bexagliflozin after 30 minutes in a solution of 0.1 N HCl at 37±0.5° C. in an Apparatus 2 (a paddle apparatus) with a paddle speed of about 75 rpm

In some embodiments, the invention also provides an oral dosage form that produces in a cohort of healthy feline subjects a geometric mean Cₘₐₓ and geometric mean AUC₀₋ₜ for which the 90% confidence intervals of the log-transformed Cₘₐₓ and log-transformed AUC₀₋ₜ fall, upon exponentiation, completely within the range 800.00-1250.00% of the geometric mean Cₘₐₓ and geometric mean AUC₀₋ₜ, respectively, of the values produced in the same cohort by reference formulations.

In some embodiments, the invention also provides a solid oral dosage form that produces in a cohort of healthy subjects a geometric mean Cₘₐₓ and geometric mean AUC₀₋ₜ for which the 90% confidence intervals of the log-transformed Cₘₐₓ and log-transformed AUC₀₋ₜ fall, upon exponentiation, completely within the range 80.00-125.00% of the geometric mean Cₘₐₓ and geometric mean AUC₀₋ₜ, respectively, of the values produced in the same cohort by a reference tablet having one of the following compositions:
(a) A tablet consisting of an admixture of 15 mg bexagliflozin, 135 mg lactose monohydrate, e.g., Foremost NF lactose hydrate, modified spray dried, 154.5 mg microcrystalline cellulose, e.g., Heweten 102, 37.5 mg of an appropriate palatant, e.g., FlavorPAL™ X1212.1, 24 mg of pregelatinized starch, e.g., Colorcon Starch 1500®, 6 mg of amorphous anhydrous colloidal silicon dioxide, e.g., Aerosil 200 Pharma, and 3 mg magnesium stearate, e.g., Hyqual®; where the core has a tablet hardness of between 5 and 10 kp and is formed by compression using a 10 mm pentagonal-shaped tablet punch.
(b) A tablet consisting of an admixture of 15 mg bexagliflozin, 22.5 mg lactose monohydrate, e.g., Foremost NF lactose hydrate, modified spray dried, 25.5 mg microcrystalline cellulose, e.g., Heweten 102, 6 mg of an appropriate palatant, e.g., FlavorPAL™ X1212.1, 4 mg of pregelatinized starch, e.g., Colorcon Starch 1500®, 2 mg of amorphous anhydrous colloidal silicon dioxide, e.g., Aerosil 200 Pharma, and 1 mg magnesium stearate, e.g., Hyqual®; where the core has a tablet hardness of between 5 and 10 kp and is formed by compression using a 5-6 mm round-shaped tablet punch.

The invention thus provides oral dosage forms which are bioequivalent to reference tablets. The oral dosage form will include the same amount of bexagliflozin as the relevant reference tablet.

The invention also provides reference oral solution dosage forms. The reference oral solution dosage form includes the same amount of bexagliflozin as the reference tablet in 0.5 mL of solution and will produce the same exposure, measured as AUC, as the reference tablet following delivery of 0.5 mL of solution and has the following composition per mL: bexagliflozin, 30 mg, ethanol, 150 μL, glycerol, 250 μL, PEG-400, 150 μL, polysorbate 80, 50 μL, palatant, 2 to 5 μL and phosphate buffered saline to 1 mL volume (approximately 365 μL). Phosphate buffered saline contains 137 mM NaCl, 2.7 mM KCl, 8 mM Na₂HPO₄, and 2 mM KH₂PO₄.

It is well known in the bioavailability and bioequivalence arts how to determine whether any particular dosage unit meets regulatory requirements for equivalent bioavailability and pharmacokinetic bioequivalence, e.g., see: Niazi (2014) Handbook of Bioequivalence Testing, 2^{nd} Edition, ISBN 978-1482226379; FDA Guidance for Industry #35 Bioequivalence Guidance (November, 2006), FDA Guidance for Industry Bioequivalence: Blood Level Bioequivalence Study, (December 2016); FDA Guidance for Industry Bioavailability and Bioequivalence Studies Submitted in NDAs or INDs—General Considerations (March 2014); and Guideline on the Investigation of Bioequivalence, EMA January 2010 (CPMP/EWP/QWP/1401/98 Rev. 1/Corr**).

An important element of any bioequivalence study is the assay used to determine the plasma concentration of the target analyte, and in general the recommendations of FDA Guidance for Industry: Bioanalytical Method Validation (May 2018) should be followed to ensure that the methods followed and data obtained conform to regulatory expectations for quality.

Many factors that vary from one individual member of a species to another can affect the concentration of a drug in plasma. It is therefore common to take into account the mass of the subject, whether the drug is administered in the fasted or fed state, the diet and medications of the subject, and the signalment of the subject. As such, drug concentrations can vary substantially from one subject to another, even under optimally controlled conditions. To control for inter-subject variation as much as possible, formulations to be compared are often administered to young healthy adult subjects in the fasted state that have been provided a standard diet and housing conditions. When reference is made to properties that are measured in vivo, it is appropriate to adjust or normalize the effects to the expected behavior in a well-characterized prototypical subject.

From a practical perspective, though, even the specification of a prototypical subject cannot capture all of the variation between individuals, and for this reason, comparisons between formulations are typically performed by administering to the same individual each of the formulations to be compared, for example the reference formulation on one day and the comparator formulation on another, and vice versa. Usually a substantial period of time (at least ten half-lives of the drug from the preceding formulation) is allowed to elapse so that prior administration of one formulation has little likelihood of affecting measurements made after the administration of the subsequent formulation. Because substantial inter-individual variation is nearly always present, the comparisons are usually made on groups of individuals, typically no fewer than 12. When certain criteria are met for the comparison of the pharmacokinetic measurements between the subjects who had received each of the two formulations, the formulations are said to be bioequivalent.

There are in principle many ways to define bioequivalence between formulations as discussed in FDA Guidance for Industry #35, Bioequivalence Guidance (November 2008) but the most common is by blood level equivalence. According to this standard two preparations can be considered bioequivalent for a particular pharmacokinetic parameter if the lower bound of the 90% confidence interval for the logarithm of the geometric mean for the parameter for a test formulation yields a value upon exponentiation that is ≥some value, usually 80.00%, of the geometric mean for the same parameter for the reference formulation and if the upper bound of the 90% confidence interval for the logarithm of the geometric mean of the parameter for the test formulation yields a value upon exponentiation that is ≤another value, usually 125.00%, of the geometric mean for the parameter for the reference formulation. The typical parameters that must be found to meet this test are the observed maximum drug concentration (Cₘₐₓ), the area under the curve for the concentration as a function of time from the beginning of dosing to the last accurately measurable value (AUC₀₋ₜ) and the area under the curve for the concentration as a function of time from the beginning of dosing, extrapolated to infinite time (AUC_{0-∞}). Geometric means and logarithms are used in these calculations because most physiological variables, including drug plasma concentrations, typically show a log-normal distribution on repeated sampling of the same individual, and on sampling from different individuals within a population.

In one aspect, the invention therefore provides a tablet comprising bexagliflozin, wherein the tablet is bioequivalent by Cₘₐₓ and AUC₀₋ₜ with any one of reference tablets (a) or (b) described above.

To ensure statistical power a study to measure the Cₘₐₓ and AUC₀₋ₜ values will be performed in multiple subjects, e.g., in a group of at least 12 (and normally between 24 and 36) healthy adults.

For establishing bioequivalence a two-period, two-sequence, two-treatment, single-dose, crossover study design can be used, a single-dose parallel study design, or a replicate study design. The preferred design is a two-period, two-sequence, two-treatment, single-dose, crossover study using healthy subjects. Each study subject should receive each treatment (test and reference drug) in random order. The most accurate, sensitive and reproducible method of measuring the drug concentration in plasma should be used. For bexagliflozin the preferred method is a validated high performance or ultra high performance liquid chromatographic (HPLC or UPLC) separation with detection of the analyte by a tandem mass spectrometry method.

A minimum of 12 subjects with evaluable data are generally required to support a determination of bioequivalence. For a study conducted in the fasted prandial state, a minimum fast of 8 h before dosing is required and water should be withheld from 1 h before to 1 h after dosing. Food should not be provided for at least 4 h following dosing.

Venous blood specimens should be drawn at appropriate intervals, generally consisting of 12 to 18 specimens in total, and covering at least three terminal elimination half-lives of the drug. Dense sampling around the expected Tₘₐₓ is recommended to provide the most accurate Cₘₐₓ.

Because determining the Cₘₐₓ and AUC₀₋ₜ values necessarily consumes each tablet tested, and because variation would be present from one test to the next, even if the tablets were identical in all respects and the same subject were used, the pharmacokinetic parameters are determined for an average of the Cₘₐₓ and AUC values of a collection of subjects dosed with a representative sample set of tablets chosen substantially at random from a manufacturing batch. The average is composed geometrically instead of arithmetically. To take the Cₘₐₓ as an example in this and the following, for a cohort of six subjects, the geometric mean Cₘₐₓ is calculated as the sixth root of the product of the six Cₘₐₓ values for the subjects. The same result will be obtained if the arithmetic average of the logarithms of the Cₘₐₓ values is exponentiated. The values for the logarithms of the Cₘₐₓ for each subject will collectively create a distribution of individual logarithms of Cₘₐₓ values.

To compare a second manufacturing batch to the first, the measurement process can be repeated with the same subjects but with tablets from the second manufacturing batch. (In actual practice, the order of administration would typically be randomly chosen for each subject, so that some would receive tablets from the second manufacturing batch first and some from the first manufacturing batch first.) For each subject a difference is calculated by subtracting the logarithm of the Cₘₐₓ for the tablet from the first manufacturing batch from the logarithm of the Cₘₐₓ for the tablet from the second manufacturing batch. The exponential of this difference is the ratio of the Cₘₐₓ for the second tablet to the Cₘₐₓ for the first tablet, which is unity if the difference is zero (e⁰=1). Following the usual statistical method for analyzing differences between two collections of values (analysis of variance), the endpoints of the 90% confidence interval for the differences of the logarithms are determined. For the two distributions to be considered bioequivalent, the endpoints of the 90% confidence interval for the differences of the logarithms must fall between the prespecified values appropriate to the regulatory standard, for example −0.22314 and +0.22314. If these values are exponentiated they give 80.00% and 125.00% respectively (e.g., e^{-0.22314}=0.8000). Other endpoint values as determined by regulatory agencies may be appropriate, for example −0.28768 to +0.28768, corresponding to 75.00% to 133.33% or −0.51083 and +0.51083, corresponding to 60.00% and 166.67%.

Although it is considered advantageous to dose each subject with tablets from each manufacturing batch to minimize variation between the measured values, if different cohorts of subjects are used for evaluating the tablets from the two manufacturing batches a similar approach can be used in which the mean difference in the logarithms for the two cohorts is calculated and a 90% confidence interval for the differences of the logarithms is constructed.

This type of test can be applied to establish whether tablets in question are tablets as defined herein. If a batch of tablets made by an unknown manufacturing process is compared by the methodology described above to a batch of tablets of the present invention defined by reference to Cₘₐₓ and AUC₀₋ₜ, and for both the Cₘₐₓ and the AUC₀₋ₜ the endpoints of the 90% confidence interval for the differences of the logarithms of the values for the two batches falls between the prespecified values, the batch of tablets made by the unknown process are tablets which meet the relevant Cₘₐₓ and AUC₀₋ₜ requirements.

A corollary of the above is that if a cohort of subjects is dosed twice with tablets of the present invention from the same manufacturing batch, and defined by reference to Cₘₐₓ and AUC₀₋ₜ, the endpoints of the 90% confidence interval for the differences of the logarithms between the values for the first and second dosing for both the Cₘₐₓ and the AUC₀₋ₜ will fall between the prespecified values.

This can be expressed more formally to state that two representative sample sets from the same batch will produce in a cohort of healthy subjects an inter-set mean difference in the logarithm of the Cₘₐₓ and the logarithm of the AUC₀₋ₜ for which the endpoints of the 90% confidence interval for the inter-set differences of the logarithms falls between the prespecified values. The distinction from the preceding paragraph is that the order of testing from the two sample sets may be randomly assigned among the subjects of the cohort, as for example is recommended in bioequivalence testing regulatory guidance documents.

Methods for the dissolution testing of solid oral dosage forms are well known in the art and include USP <711>, which specifies the types of apparatus as well as the methods for their use.

Testing for bexagliflozin veterinary tablets is conducted in USP Apparatus 2 (a paddle apparatus, e.g., with a nominal capacity of 1 liter), without sinkers, charged with 500 mL of 0.1 N HCl (i.e., simulated gastric fluid) and stirred at a rate of 75 rpm with the temperature maintained at 37±0.5° C. Individual tablets are placed in the apparatus and sampling conducted at the specified times (e.g., 5, 10, 15, 20, 25, 30, 45 and 60 min, followed by 15 minutes of additional agitation at 250 rpm) by withdrawal of 1 mL of fluid without replacement. At each timepoint the concentration of bexagliflozin in the fluid sample is determined (e.g., by a validated HPLC method), thereby permitting calculation of the amount which has been released from a tablet. These conditions conform to the recommendations of FDA Guidance for Industry Dissolution Testing and Acceptance Criteria for Immediate-Release Solid Oral Dosage Form Drug Products Containing High Solubility Drug Substances (August 2018), with the use of an angular velocity of 75 rpm justified by the necessity to avoid coning (id., IV. Standard Dissolution Testing Conditions).

Testing can proceed in up to three stages, as described in USP General Chapter <711> Dissolution, Acceptance Table 1. For bexagliflozin veterinary tablets, the quantity Q, the amount of dissolved active ingredient, is 80%. For avoidance of doubt a Q of 80% means that 12 mg of bexagliflozin have dissolved in the simulated gastric fluid. In the first stage, six tablets are analyzed. A success is recorded if each unit is not less than Q+5% (85%). If this criterion is not met, in stage 2 an additional 6 tablets are analyzed. A success is recorded: (i) if the average of the 12 units (from stages 1 and 2) is equal to or greater than Q, and (ii) no unit is less than Q−15% (65%). If the stage two criteria are not met, stage three testing must be undertaken. At stage three an additional 12 tablets are tested and the cumulative data for the 24 tablets tested must satisfy the following conditions: (i) the average of all 24 tablets must be equal to or greater than Q (80%); (ii) not more than 2 of the 24 tablets are less than Q−15% (65%); and (iii) no tablet is less than Q−25% (55%).

A manufacturing batch of bexagliflozin veterinary tablets is said to have passed formal dissolution acceptance testing if the criteria for success for at least one of the three testing stages is satisfied. Representative units of the manufacturing batch will meet these criteria, as defined in Acceptance Table 1 of USP <711>. In practical terms, testing is terminated once a success has been achieved. Additional testing, for example repeat testing to begin anew at stage one if testing fails at stage three, should not be performed.

The invention thus provides a tablet comprising bexagliflozin, wherein the tablet is from a manufacturing batch having a composition or method of testing or manufacture that falls within the formal acceptable ranges for process, testing or ingredient variation of the tablets (a) or (b) above.

Similarly, the invention provides a solid oral dosage form (and in particular a tablet, such as a flavored veterinary tablet) that contains bexagliflozin and that in an in vitro dissolution test in simulated gastric fluid has a f₂ value of >50 when compared to one of reference tablets (a) or (b) as defined above, wherein f₂ is the decimal logarithmic reciprocal square root transformation of the sum of the squared error: f₂=100−25 log₁₀(1+n⁻¹Σₗ₌₁ⁿ(Rᵢ−Tᵢ)²) where: n is number of time points at which dissolution is measured; Rᵢ is the dissolution percentage of the reference tablet at the i-th timepoint; and Tᵢ is the dissolution percentage of the test solid oral dosage form at the i-th timepoint.

The invention also provides a solid oral dosage form, typically a veterinary tablet, that contains bexagliflozin and that passes formal dissolution acceptance testing (see above) in simulated gastric fluid with criterion standards for release of >41.2% of the bexagliflozin dosage after 5 minutes and ≥80% of the bexagliflozin after 30 minutes. Preferably, the criterion standard for dissolution acceptance testing requires that between 65.4-78.4% of the bexagliflozin be released after 10 minutes and/or between 80.1-86.2% of the bexagliflozin be released after 15 minutes. In the formal dissolution acceptance testing, these dosage forms pass at least one level of a formal three level testing protocol as defined by USP <711> Acceptance Table 2.

A. Palatants

It is well known that companion animals, particularly felines, can be difficult to dose with pharmaceutical agents. Palatants are excipients that are added to a formulation to enhance the palatability of the formulation to a companion animal, thereby improving the success rate of dosing.

Palatants of the present disclosure include flavors that are attractive to companion animals such as sweet palatants or meat palatants. Sweet palatants are typically used for dogs or other non-feline companion animals because felines cannot perceive sweet flavors.

In an embodiment, the palatant comprises tuna, salmon, cream, beef, peanut, catnip, chicken liver powder, poultry extract, avian hydrolyzed liver, butter, or bacon flavoring. In another embodiment, the palatant comprises chicken liver flavoring. In another embodiment, the palatant comprises hydrolyzed chicken liver. In another embodiment, the palatant comprises avian hydrolyzed liver. In another embodiment, the palatant comprises peanut butter flavoring.

The formulations described herein are suitable for administration to cats, dogs, and other companion animals. The formulations can be successfully dosed with very few dosing rejections. For example, in some embodiments the formulation can be delivered daily with fewer than 1 dosing rejection per 30 dosing events. In some embodiments, the formulation can be delivered daily with fewer than 1 dosing rejection per 100 dosing events. In some embodiments, the formulation can be delivered daily with fewer than 1 dosing rejection per 300 dosing events.

B. Tablet Formulation Components

The tablet formulations of the present disclosure can include at least one ingredient selected from one or more fillers, one or more glidants, one or more lubricants, and one or more binders, or other ingredients.

In some embodiments, the tablet formulations of the present disclosure include about 1 to 25%, 4 to 20%, 4 to 8%, 2 to 6%, 8 to 16, or 17 to 23% by weight of bexagliflozin. In some embodiments, the tablet formulations of the present disclosure include about 20% by weight of bexagliflozin In some embodiments, the tablet formulations of the present disclosure include about 4% by weight of bexagliflozin.

In some embodiments, the tablet formulations of the present disclosure also include a palatant. In some embodiments, the tablet formulations of the present disclosure include about 1 to 30%, 2 to 25%, 5 to 15%, or 7 to 10% by weight of palatant. In some embodiments, the tablet formulations of the present disclosure include about 7.9% by weight of palatant. In some embodiments, the tablet formulations of the present disclosure include about 10% by weight of palatant. Suitable palatants are further described in the section above.

In some embodiments, the tablet formulations of the present disclosure include one or more fillers. Suitable fillers are described below. In some embodiments, the one or more fillers are present in an amount of about 1 to 70, 5 to 65, 20 to 60, 30 to 50, or 31-45% by weight. In some embodiments, one or more fillers are present in about 34% by weight. In some embodiments, one or more fillers are present in about 41% by weight.

In some embodiments, tablet formulations of the present disclosure include one to three fillers. In some embodiments, tablet formulations of the present disclosure include one to two fillers. In some embodiments, tablet formulations of the present disclosure include two fillers.

Suitable fillers include, for example, sugar alcohols (e.g., mannitol, sorbitol, xylitol, lactitol), inorganic salts, cellulose derivatives (e.g., microcrystalline cellulose, silicified microcrystalline cellulose, cellulose, hypromellose), calcium sulfate, aluminum and magnesium silicate complexes and oxides, and the like. In some embodiments, the one or more fillers include cellulose derivatives. In some embodiments, the one or more fillers are microcrystalline cellulose.

In some embodiments, the tablet formulations of the present disclosure include one or more glidants. Suitable glidants are described below. In some embodiments, the one or more glidants are present in an amount of about 0.5 to 8, 1 to 4, or 1.5 to 3% by weight. In some embodiments, the one or more glidants are present in an amount of about 2.6% by weight. In some embodiments, the one or more glidants are present in an amount of about 1.6% by weight. In some embodiments, the one or more glidants are present in an amount of about 1, 2, 3, 4, 5, 6, 7, 8, 9, or 10% by weight. In some embodiments, the one or more glidants are present in about 5% by weight.

Glidants are substances that increase the flowability of a powder, which is helpful during tableting processes. Suitable glidants include, for example, colloidal silicon dioxide, ascorbyl palmitate, calcium palmitate, talc, magnesium carbonate, and the like. In some embodiments, the one or more glidants include colloidal silicon dioxide.

In some embodiments, the tablet formulations of the present disclosure include one or more lubricants. Suitable lubricants are described below. In some embodiments, the one or more lubricants are present in an amount of about 0.1 to 8, 0.1 to 4, 0.5 to 2, 0.6 to 1.3% by weight. In some embodiments, one or more lubricants are present in an amount of about 0.5, 0.75, 1, 1.5, 2, 3, 4, or 5% by weight. In some embodiments, one or more lubricants are present in an amount of about 1.3% by weight. In some embodiments, one or more lubricants are present in an amount of about 0.8% by weight.

In some embodiments, tablet formulations of the present disclosure include one to three lubricants. In some embodiments, tablet formulations of the present disclosure include one lubricant.

Suitable lubricants include, for example, magnesium stearate, stearic acid, carnauba wax, hydrogenated vegetable oils, mineral oil, polyethylene glycols, and sodium stearyl fumarate. In some embodiments, the one or more lubricants is magnesium stearate.

In some embodiments, the tablet formulations of the present disclosure include one or more binders. Suitable binders are described below. In some embodiments, the one or more binders are present in an amount of about 30 to 50, 32-45, 35 to 45, or 42% by weight. In some embodiments, the one or more binders are present in an amount of about 42% by weight.

In some embodiments, tablet formulations of the present disclosure include one to three binders. In some embodiments, tablet formulations of the present disclosure include one binder. In some embodiments, tablet formulations of the present disclosure include two binders. In some embodiments the ration of a first binder to a second binder is about 36:6.

Suitable binders include, for example, povidone, lactose, starches, modified starches, pregelatinized starch, sugars, gum acacia, gum tragacanth, guar gum, pectin, wax binders, methylcellulose, carboxymethylcellulose, hydroxypropyl methylcellulose, hydroxyethyl cellulose, hydroxypropyl cellulose, copolyvidone (copovidone), gelatin, sodium alginate, and the like. Non-cellulosic binders include polymeric and other binders lacking a cellulose backbone. Examples of non-cellulosic binders include povidone, lactose, starches, modified starches, gums, guar gum, pectin, waxes, gelatins, alginates, and the like. In some embodiments, formulations contain a non-cellulosic binder such as lactose monohydrate and pregelatinized starch.

In some embodiments, a first binder comprises 29.6% to 36% of the tablet formulation by weight. In some embodiments a filler comprises 33.6% to 41% of the tablet formulation by weight. In some embodiments, a second binder comprises between 5.3 and 6.4% of the tablet formulation by weight. In some embodiments, a palatant comprises 7.9% and 10.0% of the tablet formulation by weight. In some embodiments, a glidant comprises 1 to 3% of the tablet formulation by weight. In some embodiments, a lubricant comprises 0.5 to 1.3% of the tablet formulation by weight. In some embodiments, bexagliflozin comprises 2 to 6% of the tablet formulation by weight.

In some embodiments, lactose monohydrate comprises 29.6% to 36% of the tablet formulation by weight. In some embodiments microcrystalline cellular comprises 33.6% to 42% of the tablet formulation by weight. In some embodiments, pregelatinized starch comprises between 5.3 and 6.4% of the tablet formulation by weight. In some embodiments, the palatant comprises 7.9% and 10.0% of the tablet formulation by weight. In some embodiments, silicon dioxide comprises 1 to 3% of the tablet formulation by weight. In some embodiments, magnesium stearate comprises 0.5 to 1.3% of the tablet formulation by weight. In some embodiments, bexagliflozin comprises 2 to 6% of the tablet formulation by weight.

In some embodiments, the tablet formulation described herein comprises 15 mg of bexagliflozin. In some embodiments, the tablet formulation described herein comprises 10 mg of bexagliflozin. In some embodiments, the tablet formulation described herein comprises 5 mg of bexagliflozin.

In some embodiments, the tablet formulation described herein comprises bexagliflozin, 15 mg; lactose monohydrate, between 110.0-160 mg; microcrystalline cellulose, between 117.0-185.4 mg; palatant, between 30-44.2 mg; pregelatinized starch, between 19.2-54 mg; colloidal silicon dioxide, between 6-13.5 mg; and magnesium stearate, between 3.0-3.5 mg.

In some embodiments the tablet formulation is as described in Table 34. In some embodiments, the tablet formulation is as described in Table 37.

C. Capsule Formulation Components

Capsule formulations of the present disclosure are typically loaded with a dry powder or small pellets. In some embodiments, the capsule formulation of the present disclosure are loaded with neat bexagliflozin or admixed with one or more excipients.

Additional excipients in the capsule formulations include those that can improve desirable biologic properties such as increased adsorption. In some embodiments, the capsule formulations of the present disclosure include microcrystalline cellulose. When admixed with one or more excipients, bexagliflozin can comprise 1 to 99% of the total mixture by weight. In some embodiments, bexagliflozin comprises 5% of the mixture (w/w/).

Capsules of the present disclosure are generally hard gelatin capsules; however, different materials for making capsules are known in the art and are embraced by the present disclosure.

D. Softgel Formulation Components

The softgel formulations of the present disclosure can include at least one ingredient selected from one or more solubilizers, one or more surfactants, one or more polymeric solubility enhancers.

In some embodiments, the softgel formulations of the present disclosure include about 1 to 25%, 2 to 20%, 3 to 10%, 2 to 6%, or 10 to 20% by weight of bexagliflozin. In some embodiments, the softgel formulations of the present disclosure include about 3% by weight of bexagliflozin

In some embodiments, the softgel formulations of the present disclosure also include a palatant. In some embodiments, the softgel formulations of the present disclosure include about 1 to 30%, 2 to 25%, 4 to 10%, 2 to 6, or 7 to 10% by weight of palatant. In some embodiments, the softgel formulations of the present disclosure include about 4% by weight of palatant. Suitable palatants are further described in an earlier section.

In some embodiments, the softgel formulations of the present disclosure include one or more solubilizers. Suitable solubilizers are described below. In some embodiments, the one or more solubilizers are present in an amount of about 10 to 70, 20 to 65, 40 to 60, or 45 to 50% by weight. In some embodiments, one or more solubilizers are present in about 47% by weight.

Suitable solubilizers include, for example, diethylene glycol ethyl ether (DEGEE), Labrasol, Gelucire 44/14, Labrafil M2130CS, Labrafil M2125CS, Gelucire 50/13, and the like. In some embodiments, the one or more solubilizers are diethylene glycol ethyl ether.

In some embodiments, the softgel formulations of the present disclosure include one or more surfactants. Suitable surfactants are described below. In some embodiments, the one or more surfactants are present in an amount of about 10 to 60, 25 to 50, 30 to 40, or 35 to 45% by weight. In some embodiments, the one or more surfactants are present in an amount of about 35% by weight.

Suitable surfactants include, for example, D-α-tocopherol polyethylene glycol succinate, polyoxyl 40 hydrogenated castor oil, macrogol-40-glycol hydroxystearate, macrogol glycerol ricinoleate, polysorbate 20, polysorbate 40, polysorbate 60, polysorbate 80, and polysorbate 100 and the like.

In some embodiments, the softgel formulations of the present disclosure include one or more polymeric solubility enhancers. Suitable polymeric solubility enhancers are described below. In some embodiments, the one or more binders are present in an amount of about 2 to 20, 5 to 15, 8 to 12% by weight. In some embodiments, the one or more surfactants are present in an amount of about 10% by weight.

Suitable polymeric solubility enhancers include, for example, a polyvinylpyrrolidone (povidone or PVP). In one embodiment, the polymeric solubility enhancers is selected from the group consisting of PVP K12, PVP K17, PVP K30, PVP K60, and PVP K90. In some embodiments, the polymeric solubility enhancers is PVP K90.

In some embodiments the softgel formulation is as described in Table 8. In some embodiments, the softgel formulation is as described in Table 9.

Softgel components of the present disclosure are typically gelatin-based comprising a mixture of gelatin and a plasticizer (e.g., glycerin), and optionally water or a coloring agent.

E. Liquid Formulation Components

Liquid formulations of the present disclosure generally include components that are in their liquid form at room temperature and dissolve bexagliflozin. Generally, liquid formulations can be prepared using ethanol-glycerol mixtures further comprising additional components. Liquid formulations can also be prepared using diethylene glycol ethyl ether (DEGEE)-PEG-400 mixtures.

Buffers are also optionally included in a liquid formulations to maintain a desired pH. Suitable buffers include, for example, PBS, citrate, fumarate, or combinations thereof. Typically the amount of buffer is added is the amount sufficient to achieve and maintain the desired pH or the complete the desired volume.

In some embodiments, liquid formulations of the present disclosure comprise diethylene glycol ethyl ether (DEGEE), PEG-400, bexagliflozin and, a buffer. The amount of bexagliflozin in these solutions can vary. Exemplary amounts are from 1 to 50 mg. In some embodiments, the liquid formulation comprises 3 mg, 10 mg, or 30 mg of bexagliflozin. In some embodiments the concentration of bexagliflozin in the liquid formulation is about 100 mM. In some embodiments, the ratio of DEGEE to PEG-400 is between 4:1 and 1:4. In some embodiments, the ratio of DEGEE to PEG-400 is between 2:3 and 3:2. In some embodiments, the ratio of DEGEE to PEG-400 is between 1:2 and 2:1. In some embodiments, the ratio of DEGEE to PEG-400 is 3:2. In some embodiments the buffer is fumarate buffer pH 6.5. In some embodiments the liquid formulation is as described in Table 4.

In some embodiments, liquid formulations of the present disclosure comprise ethanol, glycerol, bexagliflozin, a buffer, and optionally further components. The amount of bexagliflozin in these solutions can vary. Exemplary amounts are from 1 to 50 mg. In some embodiments, the liquid formulation comprises 3 mg, 10 mg, or 30 mg of bexagliflozin. In some embodiments, the ratio of ethanol to glycerol is between 4:1 and 1:4. In some embodiments, the ratio of ethanol to glycerol is between 5:3 and 3:5. In some embodiments, the ratio of ethanol to glycerol is between 4:3 and 3:4. In some embodiments, the ratio of ethanol to glycerol is between 1:2 and 2:1. In some embodiments, the ratio of ethanol to glycerol is 3:4. In some embodiments, the ratio of ethanol to glycerol is 1:2. In some embodiments, the ratio of ethanol to glycerol is 3:5. In some embodiments, the buffer is PBS pH 7.4 or citrate buffer pH 6.5 or fumarate buffer pH 6.5. Further components in these liquid solutions include one or more of propylene glycol, PEG-400, polysorbate 80, sucralose, sorbitol, 2-pyrrolidone.

In some embodiments, the liquid formulations of the present disclosure also include a palatant. Typically, the amount of palatant added to the liquid formulation is a sufficient amount to reduce dosing rejections from the companion animal. The palatant for the liquid formulation can be provided in solid form and dissolved in the liquid formulation, or pre-dissolved in a concentrated stock solution and added to the liquid formulation. Suitable palatants are further described in an earlier section. In some embodiments, the palatant comprises cream, tuna, salmon, or bacon flavoring.

In some embodiments, the liquid formulations of the present disclosure include about 0.01 to 5% by volume of palatant. In some embodiments, the liquid formulations of the present disclosure include about 0.1 to 15, 0.1 to 5 mg/mL, 1 to 3, 4 to 9, or 10 to 5 mg/mL of palatant. In some embodiments, the liquid formulations of the present disclosure include about 1 to 15 mg/mL of palatant.

In some embodiments, the liquid formulation further comprises a preservative. In some embodiments, the preservative is butylated hydroxyanisole, methyl paraben, ethyl paraben, propyl paraben, butyl paraben, sodium benzoate, sorbic acid, potassium sorbate, propionic acid, and the like. In some embodiments, the liquid formulation further comprises butylated hydroxyanisole.

In some embodiments the liquid formulation is as described in Tables 2, 3, or 6. In some embodiments the liquid formulation is as described in Tables 16 or 23.

In some embodiments the liquid formulation comprises, bexagliflozin, ethanol, glycerol, PEG-4000, polysorbate, and phosphate buffered saline (PBS), pH 7.4.

In some embodiments, provided herein is a liquid formulation containing bexagliflozin, 20 to 40 mg/mL, ethanol, 125 to 175 mg/mL, glycerol, 250 to 300 mg/mL, PEG-400, 125 to 175 mg/mL, polysorbate 80, 25 to 75 mg/mL, one or more optional palatants, 1 to 15 mg/mL and with the remainder comprising an aqueous buffer with pH between 6.0 and 8.0.

In some embodiments, provided herein is a liquid formulation containing bexagliflozin, 30 mg/mL, ethanol, 125 to 175 mg/mL, glycerol, 250 to 300 mg/mL, PEG-400, 125 to 175 mg/mL, polysorbate 80, 25 to 75 mg/mL, one or more optional palatants, 1 to 15 mg/mL and with the remainder comprising an aqueous buffer with pH between 6.0 and 8.0.

In some embodiments, the liquid formulation comprises bexagliflozin, 30 mg/mL, ethanol, 150 mg/mL, glycerol, 250 to 300 mg/mL, PEG-400, 150 mg/mL, polysorbate 80, 50 mg/mL, one or more optional palatants, 1 to 15 mg/mL and with the remainder comprising phosphate buffered saline, pH 7.4.

F. Methods of Use

The present disclosure further provides methods of using the formulations of bexagliflozin described herein for the prevention and treatment of disease. In one embodiment, the present disclosure provides a method of treating a disease or condition affected by inhibiting SGLT2, the method including administering to a subject in need thereof a formulation described herein. Diseases affected by inhibiting SGLT2 include, but are not limited to, type 1 and type 2 diabetes mellitus, hyperglycemia, renal failure, diabetic complications (such as retinopathy, nephropathy, neuropathy, ulcers, micro- and macroangiopathies, gout and diabetic foot disease), insulin resistance, metabolic syndrome (Syndrome X), hyperinsulinemia, hypertension, hyperuricemia, cardiac failure, obesity, edema, dyslipidemia, chronic heart failure, atherosclerosis, cancer and related diseases, which comprises administering a formulation described herein. In another embodiment the invention provides a method of using the formulations described herein, for the preparation of a medicament for treating type 1 and type 2 diabetes mellitus, hyperglycemia, diabetic complications, insulin resistance, metabolic syndrome, hyperinsulinemia, hypertension, hyperuricemia, obesity, edema, dyslipidemia, chronic heart failure, atherosclerosis, cancer and related diseases. In other embodiments, the invention provides a method of treating type 1 diabetes mellitus, type 2 diabetes mellitus, hyperglycemia, diabetic complications, insulin resistance, metabolic syndrome, hyperinsulinemia, hypertension, hyperuricemia, obesity, edema, dyslipidemia, chronic heart failure, atherosclerosis, and cancer.

In other embodiments, the present disclosure provides a method of treating diabetes, the method including administering to a subject in need thereof a formulation described herein. The diabetes can be any suitable form of diabetes, including, but not limited to, type 1 diabetes mellitus, type 2 diabetes mellitus, and diabetic complications. In some embodiments, the diabetes is type 1 diabetes mellitus. In some other embodiments, the diabetes is type 2 diabetes mellitus.

The present disclosure also contemplates the use of the formulations, in combination with other therapeutic agents, particularly those used for treating the above-mentioned diseases and conditions, such as antidiabetic agents, lipid-lowering/lipid-modulating agents, agents for treating diabetic complications, anti-obesity agents, antihypertensive agents, antihyperuricemic agents, and agents for treating chronic heart failure, atherosclerosis or related disorders. Those skilled in the art will appreciate that other therapeutic agents discussed below can have multiple therapeutic uses and the listing of an agent in one particular category should not be construed to limit in any way its usefulness in combination therapy with compounds of the present invention.

Examples of antidiabetic agents suitable for use in combination with the formulations described herein include insulin and insulin mimetics, sulfonylureas (such as acetohexamide, carbutamide, chlorpropamide, glibenclamide, glibornuride, gliclazide, glimepiride, glipizide, gliquidone, glisoxepide, glyburide, glyclopyramide, tolazamide, tolcyclamide, tolbutamide and the like), insulin secretion enhancers (such as JTT-608, glybuzole and the like), biguanides (such as metformin, buformin, phenformin and the like), sulfonylurea/biguanide combinations (such as glyburide/metformin and the like), meglitinides (such as repaglinide, nateglinide, mitiglinide and the like), thiazolidinediones (such as rosiglitazone, pioglitazone, isaglitazone, netoglitazone, rivoglitazone, balaglitazone, darglitazone, CLX-0921 and the like), thiazolidinedione/biguanide combinations (such as pioglitazone/metformin and the like), oxadiazolidinediones (such as YM440 and the like), peroxisome proliferator-activated receptor (PPAR)-gamma agonists (such as farglitazar, metaglidasen, MBX-2044, GI 262570, GW1929, GW7845 and the like), PPAR-alpha/gamma dual agonists (such as muraglitazar, naveglitazar, tesaglitazar, peliglitazar, JTT-501, GW-409544, GW-501516 and the like), PPAR-alpha/gamma/delta pan agonists (such as PLX204, GlaxoSmithKline 625019, GlaxoSmithKline 677954 and the like), retinoid-x receptor (RXR) agonists (such as ALRT-268, AGN-4204, MX-6054, AGN-194204, LG-100754, bexarotene and the like), alpha-glucosidase inhibitors (such as acarbose, miglitol and the like), stimulants of insulin receptor tyrosine kinase (such as TER-17411, L-783281, KRX-613 and the like), tripeptidyl peptidase II inhibitors (such as UCL-1397 and the like), dipeptidyl peptidase IV inhibitors (such as sitagliptin, vildagliptin, denagliptin, saxagliptin, alogliptin, dutogliptin, NVP-DPP728, P93/01, P32/98, FE 99901, TS-021, TSL-225, GRC8200, compounds described in U.S. Pat. Nos. 6,869,947; 6,727,261; 6,710,040; 6,432,969; 6,172,081; 6,011,155 and the like), glucokinase activators (such as ARRY-403, piragliatin (RO4389620), RO0281675, MK-0941, TTP355, GKA50, GKA60, GKM-001, PSN010, PSN-GK1, compounds described in Sarabu, R., et al., Expert Opinion on Therapeutic Patents, Vol. 21, No. 1, 2011, pp. 13-33, and the like), protein tyrosine phosphatase-1B inhibitors (such as KR61639, IDD-3, PTP-3848, PTP-112, OC-86839, PNU-177496, compounds described in Vats, R. K., et al., Current Science, Vol. 88, No. 2, 25 Jan. 2005, pp. 241-249, and the like), glycogen phosphorylase inhibitors (such as NN-4201, CP-368296 and the like), glucose-6-phosphatase inhibitors, fructose 1,6-bisphosphatase inhibitors (such as CS-917, MB05032 and the like), pyruvate dehydrogenase inhibitors (such as AZD-7545 and the like), imidazoline derivatives (such as BL11282 and the like), hepatic gluconeogenesis inhibitors (such as FR-225659 and the like), D-chiroinositol, glycogen synthase kinase-3 inhibitors (such as compounds described in Vats, R. K., et al., Current Science, Vol. 88, No. 2, 25 Jan. 2005, pp. 241-249, and the like), 11 beta-hydroxysteroid dehydrogenase type 1 inhibitors (such as carbenoxolone, INCB13739 and the like), glucagon receptor antagonists (such as BAY-27-9955, NN-2501, NNC-92-1687 and the like), glucagon-like peptide-1 (GLP-1), GLP-1 receptor agonists (such as exenatide, liraglutide, semaglutide, dulaglutide, CJC-1131, AVE-0100, AZM-134, LY-315902, GlaxoSmithKline 716155 and the like), or combinations of GLP-1 receptor agonists with other peptide hormone agonists or antagonists such as the dual GLP-1 GIP agonist tirzepatide or the GLP-1 agonist GIP antagonist AMG133, amylin, amylin analogs and agonists (such as pramlintide and the like), fatty acid binding protein (aP2) inhibitors (such as compounds described in U.S. Pat. Nos. 6,984,645; 6,919,323; 6,670,380; 6,649,622; 6,548,529 and the like), beta-3 adrenergic receptor agonists (such as solabegron, CL-316243, L-771047, FR-149175 and the like), and other insulin sensitivity enhancers (such as reglixane, ONO-5816, MBX-102, CRE-1625, FK-614, CLX-0901, CRE-1633, NN-2344, BM-13125, BM-501050, HQL-975, CLX-0900, MBX-668, MBX-675, 5-15261, GW-544, AZ-242, LY-510929, AR-H049020, GW-501516 and the like).

Examples of agents for treating diabetic complications suitable for use in combination with the formulations described herein include aldose reductase inhibitors (such as epalrestat, imirestat, tolrestat, minalrestat, ponalrestat, zopolrestat, fidarestat, ascorbyl gamolenate, ADN-138, BAL-ARI8, ZD-5522, ADN-311, GP-1447, IDD-598, risarestat, zenarestat, methosorbinil, AL-1567, M-16209, TAT, AD-5467, AS-3201, NZ-314, SG-210, JTT-811, lindolrestat, sorbinil and the like), inhibitors of advanced glycation end-products (AGE) formation (such as pyridoxamine, OPB-9195, ALT-946, ALT-711, pimagedine and the like), AGE breakers (such as ALT-711 and the like), sulodexide, 5-hydroxy-1-methylhydantoin, insulin-like growth factor-I, platelet-derived growth factor, platelet-derived growth factor analogs, epidermal growth factor, nerve growth factor, uridine, protein kinase C inhibitors (such as ruboxistaurin, midostaurin and the like), sodium channel antagonists (such as mexiletine, oxcarbazepine and the like), nuclear factor-kappaB (NF-kappaB) inhibitors (such as dexlipotam and the like), lipid peroxidase inhibitors (such as tirilazad mesylate and the like), N-acetylated-alpha-linked-acid-dipeptidase inhibitors (such as GPI-5232, GPI-5693 and the like), and carnitine derivatives (such as carnitine, levacecamine, levocarnitine, ST-261 and the like).

Examples of antihyperuricemic agents suitable for use in combination with the formulations described herein include uric acid synthesis inhibitors (such as allopurinol, oxypurinol and the like), uricosuric agents (such as probenecid, sulfinpyrazone, benzbromarone and the like) and urinary alkalinizers (such as sodium hydrogen carbonate, potassium citrate, sodium citrate and the like).

Examples of lipid-lowering/lipid-modulating agents suitable for use in combination with the formulations described herein include hydroxymethylglutaryl coenzyme A reductase inhibitors (such as acitemate, atorvastatin, bervastatin, carvastatin, cerivastatin, colestolone, crilvastatin, dalvastatin, fluvastatin, glenvastatin, lovastatin, mevastatin, nisvastatin, pitavastatin, pravastatin, ritonavir, rosuvastatin, saquinavir, simvastatin, visastatin, SC-45355, SQ-33600, CP-83101, BB-476, L-669262, S-2468, DMP-565, U-20685, BMS-180431, BMY-21950, compounds described in U.S. Pat. Nos. 5,753,675; 5,691,322; 5,506,219; 4,686,237; 4,647,576; 4,613,610; 4,499,289 and the like), fibric acid derivatives (such as gemfibrozil, fenofibrate, bezafibrate, beclobrate, binifibrate, ciprofibrate, clinofibrate, clofibrate, etofibrate, nicofibrate, pirifibrate, ronifibrate, simfibrate, theofibrate, AHL-157 and the like), PPAR-alpha agonists (such as GlaxoSmithKline 590735 and the like), PPAR-delta agonists (such as GlaxoSmithKline 501516 and the like), acyl-coenzyme A:cholesterol acyltransferase inhibitors (such as avasimibe, eflucimibe, eldacimibe, lecimibide, NTE-122, MCC-147, PD-132301-2, C1-1011, DUP-129, U-73482, U-76807, TS-962, RP-70676, P-06139, CP-113818, RP-73163, FR-129169, FY-038, EAB-309, KY-455, LS-3115, FR-145237, T-2591, J-104127, R-755, FCE-27677, FCE-28654, YIC-C8-434, CI-976, RP-64477, F-1394, CS-505, CL-283546, YM-17E, 447C88, YM-750, E-5324, KW-3033, HL-004 and the like), probucol, thyroid hormone receptor agonists (such as liothyronine, levothyroxine, KB-2611, GC-1 and the like), cholesterol absorption inhibitors (such as ezetimibe, SCH48461 and the like), lipoprotein-associated phospholipase A2 inhibitors (such as rilapladib, darapladib and the like), microsomal triglyceride transfer protein inhibitors (such as CP-346086, BMS-201038, compounds described in U.S. Pat. Nos. 5,595,872; 5,739,135; 5,712,279; 5,760,246; 5,827,875; 5,885,983; 5,962,440; 6,197,798; 6,617,325; 6,821,967; 6,878,707 and the like), low density lipoprotein receptor activators (such as LY295427, MD-700 and the like), lipoxygenase inhibitors (such as compounds described in WO 97/12615, WO 97/12613, WO 96/38144 and the like), carnitine palmitoyl-transferase inhibitors (such as etomoxir and the like), squalene synthase inhibitors (such as YM-53601, TAK-475, SDZ-268-198, BMS-188494, A-87049, RPR-101821, ZD-9720, RPR-107393, ER-27856, compounds described in U.S. Pat. Nos. 5,712,396; 4,924,024; 4,871,721 and the like), nicotinic acid derivatives (such as acipimox, nicotinic acid, ricotinamide, nicomol, niceritrol, nicorandil and the like), bile acid sequestrants (such as colestipol, cholestyramine, colestilan, colesevelam, GT-102-279 and the like), sodium/bile acid cotransporter inhibitors (such as 264W94, S-8921, SD-5613 and the like), and cholesterol ester transfer protein inhibitors (such as torcetrapib, JTT-705, PNU-107368E, SC-795, CP-529414 and the like).

Examples of anti-obesity agents suitable for use in combination with the formulations described herein include serotonin-norepinephrine reuptake inhibitors (such as sibutramine, milnacipran, mirtazapine, venlafaxine, duloxetine, desvenlafaxine and the like), norepinephrine-dopamine reuptake inhibitors (such as radafaxine, bupropion, amineptine and the like), serotonin-norepinephrine-dopamine reuptake inhibitors (such as tesofensine and the like), selective serotonin reuptake inhibitors (such as citalopram, escitalopram, fluoxetine, fluvoxamine, paroxetine, sertraline and the like), selective norepinephrine reuptake inhibitors (such as reboxetine, atomoxetine and the like), norepinephrine releasing stimulants (such as rolipram, YM-992 and the like), anorexiants (such as amphetamine, methamphetamine, dextroamphetamine, phentermine, benzphetamine, phendimetrazine, phenmetrazine, diethylpropion, mazindol, fenfluramine, dexfenfluramine, phenylpropanolamine and the like), dopamine agonists (such as ER-230, doprexin, bromocriptine mesylate and the like), H₃-histamine antagonists (such as impentamine, thioperamide, ciproxifan, clobenpropit, GT-2331, GT-2394, A-331440, and the like), 5-HT2c receptor agonists (such as, 1-(m-chlorophenyl)piperazine (m-CPP), mirtazapine, APD-356 (lorcaserin), SCA-136 (vabicaserin), ORG-12962, ORG-37684, ORG-36262, ORG-8484, Ro-60-175, Ro-60-0332, VER-3323, VER-5593, VER-5384, VER-8775, LY-448100, WAY-161503, WAY-470, WAY-163909, MK-212, BVT.933, YM-348, IL-639, IK-264, ATH-88651, ATHX-105 and the like (see, e.g., Nilsson B M, J. Med. Chem. 2006, 49:4023-4034)), beta-3 adrenergic receptor agonists (such as L-796568, CGP 12177, BRL-28410, SR-58611A, ICI-198157, ZD-2079, BMS-194449, BRL-37344, CP-331679, CP-331648, CP-114271, L-750355, BMS-187413, SR-59062A, BMS-210285, LY-377604, SWR-0342SA, AZ-40140, SB-226552, D-7114, BRL-35135, FR-149175, BRL-26830A, CL-316243, AJ-9677, GW-427353, N-5984, GW-2696 and the like), cholecystokinin agonists (such as SR-146131, SSR-125180, BP-3.200, A-71623, A-71378, FPL-15849, GI-248573, GW-7178, GI-181771, GW-7854, GW-5823, and the like), antidepressant/acetylcholinesterase inhibitor combinations (such as venlafaxine/rivastigmine, sertraline/galanthamine and the like), lipase inhibitors (such as orlistat, ATL-962 and the like), anti-epileptic agents (such as topiramate, zonisamide and the like), leptin, leptin analogs and leptin receptor agonists (such as LY-355101 and the like), neuropeptide Y (NPY) receptor antagonists and modulators (such as SR-120819-A, PD-160170, NGD-95-1, BIBP-3226, 1229-U-91, CGP-71683, BIBO-3304, CP-671906-01, J-115814 and the like), ciliary neurotrophic factor (such as Axokine and the like), thyroid hormone receptor-beta agonists (such as KB-141, GC-1, GC-24, GB98/284425 and the like), cannabinoid CB1 receptor antagonists (such as rimonabant, SR147778, SLV 319 and the like (see, e.g., Antel J et al., J. Med. Chem. 2006, 49:4008-4016)), melanin-concentrating hormone receptor antagonists (such as GlaxoSmithKline 803430X, GlaxoSmithKline 856464, SNAP-7941, T-226296 and the like (see, e.g., Handlon A L and Zhou H, J. Med. Chem. 2006, 49:4017-4022)), melanocortin-4 receptor agonists (including PT-15, Ro27-3225, THIQ, NBI 55886, NBI 56297, NBI 56453, NBI 58702, NBI 58704, MB243 and the like (see, e.g., Nargund R P et al., J. Med. Chem. 2006, 49:4035-4043)), selective muscarinic receptor M₁ antagonists (such as telenzepine, pirenzepine and the like), opioid receptor antagonists (such as naltrexone, methylnaltrexone, nalmefene, naloxone, alvimopan, norbinaltorphimine, nalorphine and the like), and combinations thereof.

Examples of antihypertensive agents and agents for treating chronic heart failure, atherosclerosis or related diseases suitable for use in combination with the formulations described herein include bimoclomol, angiotensin-converting enzyme inhibitors (such as captopril, enalapril, fosinopril, lisinopril, perindopril, quinapril, ramipril and the like), neutral endopeptidase inhibitors (such as thiorphan, omapatrilat, MDL-100240, fasidotril, sampatrilat, GW-660511, mixanpril, SA-7060, E-4030, SLV-306, ecadotril and the like), angiotensin II receptor antagonists (such as candesartan cilexetil, eprosartan, irbesartan, losartan, olmesartan medoxomil, telmisartan, valsartan, tasosartan, enoltasosartan and the like), endothelin-converting enzyme inhibitors (such as CGS 35066, CGS 26303, CGS-31447, SM-19712 and the like), endothelin receptor antagonists (such as tracleer, sitaxsentan, ambrisentan, L-749805, TBC-3214, BMS-182874, BQ-610, TA-0201, SB-215355, PD-180988, BMS-193884, darusentan, TBC-3711, bosentan, tezosentan, J-104132, YM-598, S-0139, SB-234551, RPR-118031A, ATZ-1993, RO-61-1790, ABT-546, enlasentan, BMS-207940 and the like), diuretic agents (such as hydrochlorothiazide, bendroflumethiazide, trichlormethiazide, indapamide, metolazone, furosemide, bumetanide, torsemide, chlorthalidone, metolazone, cyclopenthiazide, hydroflumethiazide, tripamide, mefruside, benzylhydrochlorothiazide, penflutizide, methyclothiazide, azosemide, etacrynic acid, torasemide, piretanide, meticrane, potassium canrenoate, spironolactone, triamterene, aminophylline, cicletanine, LLU-alpha, PNU-80873A, isosorbide, D-mannitol, D-sorbitol, fructose, glycerin, acetazolamide, methazolamide, FR-179544, OPC-31260, lixivaptan, conivaptan and the like), calcium channel antagonists (such as amlodipine, bepridil, diltiazem, felodipine, isradipine, nicardipen, nimodipine, verapamil, S-verapamil, aranidipine, efonidipine, barnidipine, benidipine, manidipine, cilnidipine, nisoldipine, nitrendipine, nifedipine, nilvadipine, felodipine, pranidipine, lercanidipine, isradipine, elgodipine, azelnidipine, lacidipine, vatanidipine, lemildipine, diltiazem, clentiazem, fasudil, bepridil, gallopamil and the like), vasodilating antihypertensive agents (such as indapamide, todralazine, hydralazine, cadralazine, budralazine and the like), beta blockers (such as acebutolol, bisoprolol, esmolol, propanolol, atenolol, labetalol, carvedilol, metoprolol and the like), sympathetic blocking agents (such as amosulalol, terazosin, bunazosin, prazosin, doxazosin, propranolol, atenolol, metoprolol, carvedilol, nipradilol, celiprolol, nebivolol, betaxolol, pindolol, tertatolol, bevantolol, timolol, carteolol, bisoprolol, bopindolol, nipradilol, penbutolol, acebutolol, tilisolol, nadolol, urapidil, indoramin and the like), alpha-2-adrenoceptor agonists (such as clonidine, methyldopa, CHF-1035, guanabenz acetate, guanfacine, moxonidine, lofexidine, talipexole and the like), centrally acting antihypertensive agents (such as reserpine and the like), thrombocyte aggregation inhibitors (such as warfarin, dicumarol, phenprocoumon, acenocoumarol, anisindione, phenindione, ximelagatran and the like), and antiplatelets agents (such as aspirin, clopidogrel, ticlopidine, dipyridamole, cilostazol, ethyl icosapentate, sarpogrelate, dilazep, trapidil, beraprost and the like).

The formulations described herein are also useful for treatment of glucose disorders. In some embodiments, the present invention provides methods of decreasing blood glucose in a subject in need thereof, the methods including administering to the subject a formulation described herein. In other embodiments, the present invention provides a method of lowering serum levels of fructosamine in a subject in need thereof, the method including administering to the subject a formulation described herein. In still other embodiments, the present invention provides a method of increasing the excretion of glucose in the urine of a subject in need thereof, said method comprising administering to the subject an formulation described herein.

The treatments of the present disclosure can be administered prophylactically to prevent or delay the onset or progression of a disease or condition (such as hyperglycemia), or therapeutically to achieve a desired effect (such as a desired level of serum glucose) for a sustained period of time.

All publications and patent applications cited in this specification are herein incorporated by reference as if each individual publication or patent application were specifically and individually indicated to be incorporated by reference. Any conflict between any reference cited herein and the teaching of this specification is to be resolved in favor of the latter. Similarly, any conflict between an art-recognized definition of a word or phrase and a definition of the word or phrase as provided in this specification is to be resolved in favor of the latter. Although the foregoing invention has been described in some detail by way of illustration and example for purposes of clarity of understanding, it will be readily apparent to those of ordinary skill in the art in light of the teachings of this invention that certain changes and modifications can be made thereto without departing from the spirit or scope of the appended claims. The invention will be described in greater detail by way of specific examples.

### Examples

The following examples are offered for illustrative purposes, and are not intended to limit the invention in any manner. Those of skill in the art will readily recognize a variety of noncritical parameters which can be changed or modified to yield essentially the same results.

Compositions of bexagliflozin for oral dosing in veterinary use were prepared as liquids, softgels, capsules and tablets. Candidate veterinary formulations were evaluated by multiple criteria, including pharmacokinetics, pharmacodynamics, palatability and stability.

### Pharmacokinetics Produced by Veterinary Formulations

### Overview

Table 1 provides the exposure by Cₘₐₓ and AUC₀₋ₜ for bexagliflozin following dosing of laboratory cats with various formulations at different dose levels. The exposure is normalized to the dose in mg of bexagliflozin per kg of body weight, so that Cₘₐₓ is given in ng mL⁻¹ per mg kg⁻¹ of dose and AUC₀₋ₜ is given in ng h mL⁻¹ per mg kg⁻¹ of dose. The dose-normalized values in Table 1 provide an assessment of the effectiveness of the formulation to deliver bexagliflozin to the plasma of cats dosed orally at the indicated dose levels.

The pharmacokinetic parameters determined in the experiments presented in Table 1 were mostly evaluated following dosing in the fasted state. Two tablet formulations were also tested in the fed state for comparison. In one case, involving administration of 10 tablets per cat, the effects of offering intermittent food as a reward were investigated.

Table 1 shows that within a given formulation the normalized values for AUC are generally independent of dose level, consistent with linear pharmacokinetics. Greater variability is observed for Cₘₐₓ, whereas the AUC is more consistent.

The initial capsule formulation, consisting of capsules containing bexagliflozin, microcrystalline cellulose and magnesium stearate, produced exposures by AUC₀₋ₜ that were less than 1000 ng h mL⁻¹ per mg kg⁻¹ of dose, whereas liquid formulations containing diethylene glycol mono ethyl ether all produced exposures by AUC₀₋ₜ that were greater than 1000 ng h mL⁻¹ per mg kg⁻¹ of dose. Tablet formulations produced similar but somewhat less exposure compared to the liquid DEGEE formulation. Exposures from softgels were comparable to those from capsules, with normalized AUC₀₋ₜ less than 1000 ng h mL⁻¹ per mg kg⁻¹ of dose (Table 1).

TABLE 1
Dose-Normalized Exposure by Cₘₐₓ and AUC₀₋ₜ

| | Dose | Prandial | Cₘₐₓ/ | AUC₀₋ₜ/ |
|---|---|---|---|---|
| Dosage Form | (mg kg⁻¹) | State | dose | dose ^{a} |
| Capsule, | 0.1 | Fasted | 300 | 967 |
| MCC Blend ^{b} | | | | |
| Capsule, | 0.3 | Fasted | 293 | 819 |
| MCC Blend ^{b} | | | | |
| Capsule, | 1 | Fasted | 231 | 674 |
| MCC Blend ^{b} | | | | |
| Capsule, Neat ^{c} | 3 | Fasted | 342 | 783 |
| Capsule, Neat ^{c} | 10 | Fasted | 119 | 691 |
| Liquid, F02A ^{d} | 3 | Fasted | 644 | 2007 |
| Liquid, F16D ^{d} | 3 | Fasted | 911 | 2549 |
| Liquid, F16D-E ^{e} | 1 | Fasted | 493 | 1194 |
| Liquid, DEGEE ^{e} | 0.3 | Fasted | 777 | 1530 |
| Liquid, DEGEE ^{e} | 1 | Fasted | 748 | 1816 |
| Liquid, DEGEE ^{e} | 3 | Fasted | 282 | 1329 |
| Tablet, Resin ^{f} | 0.70 | Fasted | 488 | 1264 |
| Tablet, Resin ^{f} | 1.31 | Fasted | 595 | 1505 |
| Tablet, Resin ^{f} | 3.53 | Fasted | 353 | 1352 |
| Tablet, Resin ^{f} | 10.9 | Fasted | 566 | 2245 |
| Tablet, Resin ^{g} | 5.74 | Fasted | 414 | 1637 |
| Tablet, Resin ^{g} | 5.69 | Fed | 268 | 1503 |
| Tablet, | 5.67 | Fasted | 487 | 1953 |
| Dry Blend ^{h} | | | | |
| Tablet, | 5.41 | Fed | 283 | 1262 |
| Dry Blend ^{h} | | | | |
| Tablet, | 50.8 | Fasted | 384 | 2121 |
| Dry Blend ^{h} | | | | |
| Tablet, | 50.8 | Partial ⁱ | 390 | 1992 |
| Dry Blend ^{h} | | | | |
| Tablet, | 5.6 | Fasted | 344 | 1526 |
| Resin, H+ ^{j} | | | | |
| Tablet, | 5.46 | Fasted | 395 | 1553 |
| Resin, Na+ ^{k} | | | | |
| Softgel-B ^{l} | 1.79 | Fasted | 256 | 799 |
| Softgel-D ^{l} | 1.78 | Fasted | 420 | 984 |
| Liquid-M ^{m} | 1 | Fasted | 635 | 1166 |
| Liquid-N ^{m} | 1 | Fasted | 554 | 1097 |
| Liquid-P ^{m} | 1 | Fasted | 438 | 877 |
| Liquid-Q ^{m} | 1 | Fasted | 489 | 1096 |
| Liquid-V ^{m} | 1 | Fasted | 616 | 1131 |
| Liquid-W ^{m} | 1 | Fasted | 722 | 1264 |

^{a} Area under curve from time 0 to time of last measurable concentration, in ng h mL⁻¹
^{b} 5% bexagliflozin in microcrystalline cellulose blend, size 4 gelatin capsule
^{c} Bexagliflozin only, size 4 gelatin capsule
^{d} Compositions in Table 3
^{e} Diethylene glycol ethyl ether, 30%% in 20% aqueous buffered PEG-400 (Table 4)
^{f} Initial tablet formulation prepared by slurrying with resin, drying, mixing and compressing
^{g} Study to compare fasted and fed state
^{h} Direct compression dry blend without resin
ⁱ Small amount of food offered to reward tablet consumption
^{j} Resin in protonated form, not slurried
^{k} Resin in sodium form
^{l} Compositions in Table 8 and Table 9
^{m} Compositions in Table 6

The following examples illustrate the effects of formulation on pharmacokinetics.

### Pharmacokinetics Produced by Capsule Formulations

Capsules were evaluated using cohorts of four laboratory cats that were administered test article at multiple dose levels. The test article was prepared in size 4 gelatin capsules and administered by mouth to animals that had been fasted 12 h. The capsules were loaded with 5% bexagliflozin (w/w) in microcrystalline cellulose for the 0.1, 0.3 and 1.0 mg kg⁻¹ dose levels, and with bexagliflozin alone for the 3.0 and 10.0 mg kg⁻¹ dose levels. The cats were provided with food ad libitum 2 hours after dosing.

Blood samples were collected from the acral vein at the scheduled time points. Samples of 0.1 to 0.3 mL were drawn into 1.5 mL tubes containing dipotassium EDTA as an anticoagulant. The samples were immediately mixed by gently inverting the tube approximately 8 times. Immediately following mixing, the tubes were placed on crushed ice and kept chilled until centrifugation. Samples were centrifuged within 1 hour of collection. Following centrifugation, the tubes were returned to crushed ice and supernatants transferred into microcentrifuge tubes and frozen. The tubes were stored frozen in a −20° C. freezer at the testing facility prior to shipment for analysis. Plasma bexagliflozin concentration was measured by an LC-MS/MS method. Separations were performed using an Agilent 1200 equipped with a Thermo Betasil C18 50×2.1 mm, 5 μm packing column or a Phenomenex Gemini C18 50×4.5 mm, 5 μm packing column. Analyte detection was performed with an ABI 4000 or AB Sciex QTRAP 5500 mass spectrometer.

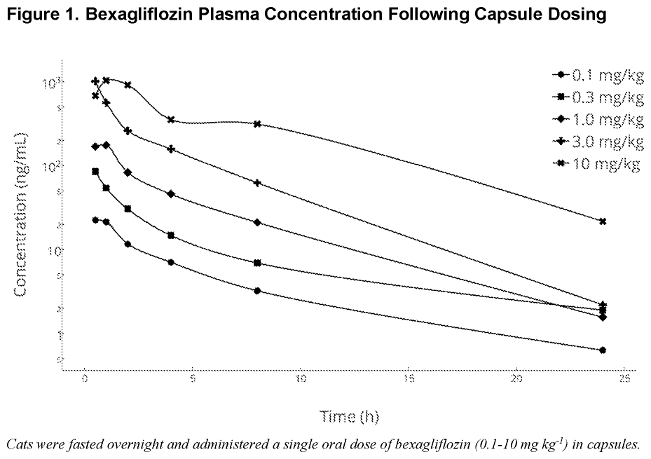

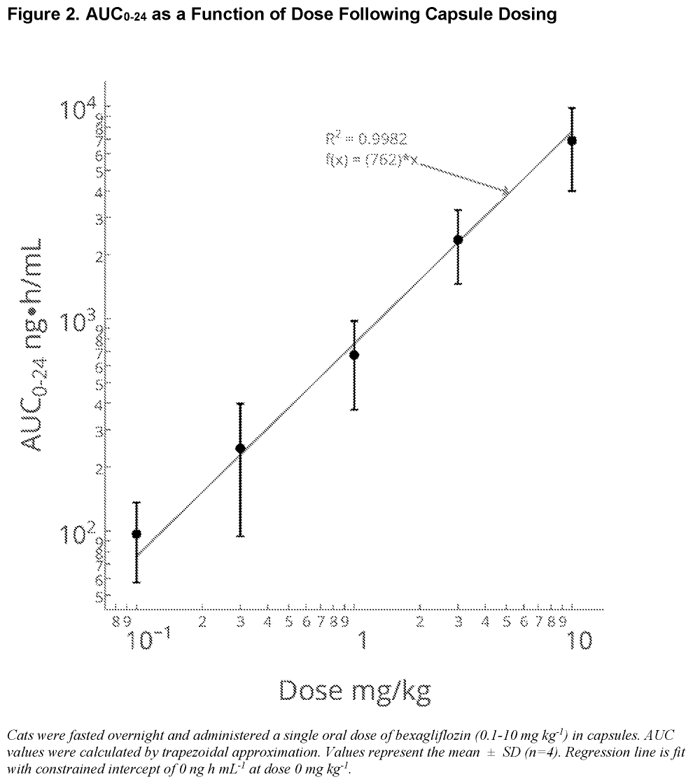

Plasma concentration profiles as a function of time are shown in FIG. 1. Bexagliflozin was rapidly absorbed with a Tₘₐₓ between 0.6 and 1.2 h. The volume of distribution was relatively large, ranging from 5.5 to 11.9 L kg⁻¹, with no dose-dependent trend. The elimination half-life ranged from 2.9 to 5.9 h, without a dose-dependent trend. The majority of the variation in half-life was attributable to variation in the volume of distribution, and the clearance was relatively constant at 1.46±0.24 L h⁻¹ kg⁻¹. The AUC₀₋₂₄ was proportional to dose over the entire dosing range and a linear regression with a constrained intercept of 0 ng h mL⁻¹ at 0 mg kg⁻¹ gave a slope of 762 ng h mL⁻¹ per mg kg⁻¹ of dose (FIG. 2). The Cₘₐₓ was dose linear to 3 mg kg⁻¹ but at 10 mg kg⁻¹ fell below the linear regression prediction.

### Pharmacokinetics Produced by Liquid Formulations

A number of liquid formulations for the delivery of bexagliflozin to cats were created and tested in various ways, including by determination of the elicited pharmacokinetics following dosing of healthy cats as described in the following.

In one example three compositions were selected for investigation (Table 2).

TABLE 2
Ethanol-Glycerol Formulation Compositions

| | Amount (mg/mL) | | |
|---|---|---|---|
| Ingredient | T-PG | F02A | F02B |
| Bexagliflozin | 30 | 30 | 30 |
| Ethanol absolute | 150 | 150 | 150 |
| Glycerol | 200 | 300 | 250 |
| Propylene glycol | 200 | — | — |
| PEG-400 | — | 150 | 150 |
| Polysorbate 80 | — | 50 | 50 |
| Sucralose | — | 5 | 5 |
| Sorbitol Solution | — | — | 150 |
| Cream Flavor | — | — | 2 |
| PBS pH 7.4 | QS | QS | — |
| Citrate Buffer pH 7.4 | — | — | QS |

QS: added as needed to desired volume

In another example, a formulation containing diethylene glycol ethyl ether (DEGEE) was created for comparison with formulation F02A. The composition of the two formulas is shown in Table 3.

TABLE 3
Glycerol Formulation
Compositions, Comparative PK

| | Amount (mg/mL) | |
|---|---|---|
| Ingredient | F16D | F02A |
| Bexagliflozin | 30 | 30 |
| Ethanol absolute | — | 150 |
| Glycerol | 200 | 300 |
| PEG-400 | — | 150 |
| Diethylene glycol ethyl ether | 300 | — |
| Polysorbate 80 | — | 50 |
| Sucralose | — | 5 |
| PBS pH 7.4 | QS | QS |

QS: added as needed to desired volume

Four laboratory cats were orally dosed with liquid formulations containing bexagliflozin at 30 mg mL⁻¹ dissolved in the aqueous solutions shown in Table 3. The dose volume was 0.1 mL kg⁻¹ to achieve a dose level of 3 mg kg⁻¹. Blood was sampled from the acral vein at pre-dose, and at 0.5, 1, 2, 4, 8 and 24 h post-dose.

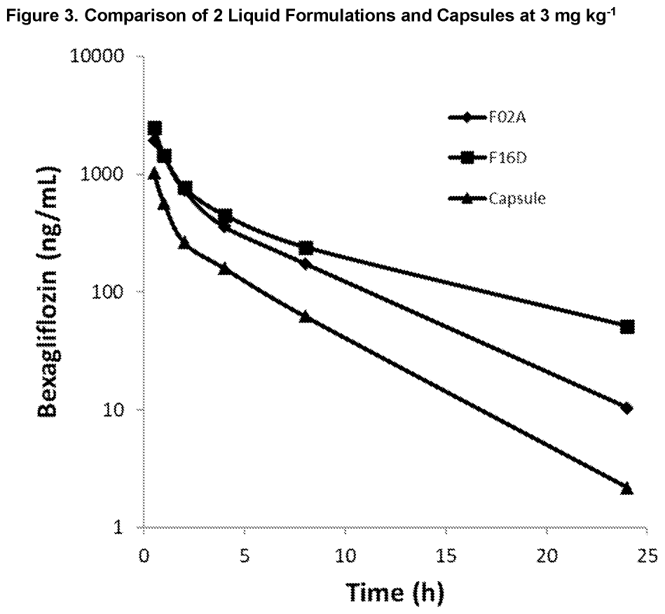

FIG. 3 shows the plasma concentration of bexagliflozin as a function of time following dosing. The initial (distribution) phase of plasma concentration decline was similar for the two liquid formulations, whereas the terminal (elimination) phase showed a slower rate of decline following dosing with the composition containing DEGEE (FIG. 3). This unusual effect suggests distribution into a different compartment following administration in the DEGEE formulation, with the most likely being a compartment proximal to, or a component of, the upper gastrointestinal tract, based on the known properties of DEGEE as a penetration enhancer.

FIG. 3 also shows the plasma concentration for bexagliflozin administered in capsules at the same dose level (3 mg kg⁻¹). The Cₘₐₓ was lower but otherwise the concentration profile closely paralleled that seen with the F02A formulation (glycerol/ethanol/PEG-400). The lower Cₘₐₓ indicates that the initial absorption was less efficient, possibly reflecting the small fluid volume and relatively low solubility of bexagliflozin (approximately 0.5 mg/mL) in aqueous media at 37° C.

In a further experiment using liquid formulations, groups of 8 or 9 cats were dosed on four consecutive days with a fixed dose of 1.5 mg of bexagliflozin on days 0 to 3, of 5 mg from days 4 to 7, and of 15 mg from days 8 to 11, administered as 0.5 mL of the formulations shown in Table 4. The fumarate buffer was prepared in the sodium form.

Blood was collected at pre-dose and at 0.5, 1, 2, 4, 8, 12 and 24 h post-dose. Blood was obtained via direct venipuncture using a suitably sized needle and syringe. Approximately 0.5 mL of blood was drawn and immediately placed into chilled tubes containing K₂EDTA as an anticoagulant. Blood tubes were inverted several times to ensure mixing of blood with anticoagulant. Blood samples were centrifuged with a force of 1100 to 1300×g for 10 min. Once separated, plasma was transferred to duplicate sets of labeled cryovials and frozen at ≤−70° C. Following in-life completion, one sample set was shipped to the bioanalytical facility on dry ice for analysis.

For analysis, 100 μL of calibration standards, QC samples, study samples, and blanks were pipetted into a 2.4-mL 96-well plate using a pipette or an automated liquid handling device. The reagent blank was 100 μL water. 50.0 μL of methanol:water (50:50 v/v) was added to wells containing blank and 50.0 μL of internal standard solution to all other wells using a pipette or an automated liquid handling device. To each well, 700 μL methyl-tert-butyl ether was added using a pipette or an automated liquid handling device. The well contents were mixed by aspirating and dispensing 25 times on an automated liquid handling device. The plate was sealed and centrifuged at a minimum of 1640×g for approximately 5 minutes. From the organic layer 500 μL was transferred to a clean 2-mL 96-well plate using an automated liquid handling device. The wells were then evaporated to dryness under a stream of nitrogen at 40° C. The dried samples were reconstituted by adding 150 μL of acetonitrile:water (30:70) using a pipette or an automated liquid handling device. The plate was sealed, vortex-mixed for 1 minute and stored under refrigerated conditions for analysis.

Plasma bexagliflozin concentration was measured by an LC-MS/MS method. Separations were performed using an Shimadzu Prominence 20 series HPLC equipped with a Thermo Aquasil C₁₈ 50×2.1 mm, 3 μm packing column. Elution was isocratic with 10 mM ammonium acetate (62%) and acetonitrile (38%) and a typical retention time of 0.85 min. Analyte detection was performed in electrospray ionization positive mode with a Sciex API 4500 mass spectrometer at a spray voltage of 4000 V. The internal standard was bexagliflozin uniformly labeled with ¹³C in the glucose moiety and detection was based on the transitions of 482.2→167.1 for the analyte and 488.2→169.1 for the standard.

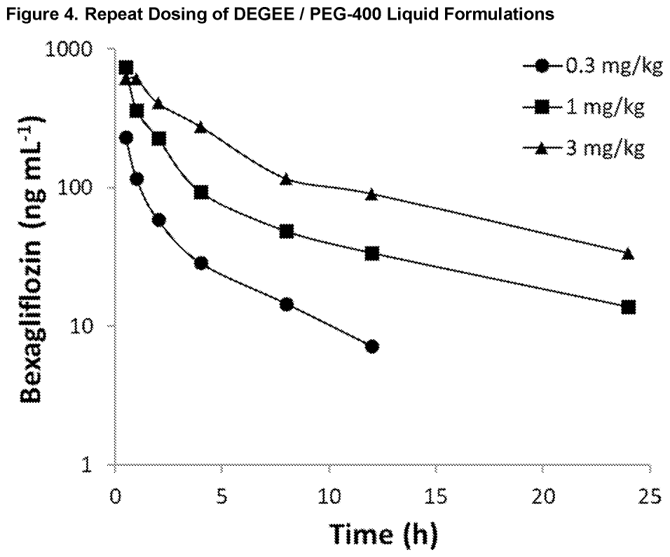

Body masses of the cats gave dosing in the mean of 0.3, 1.0 and 3.0 mg kg⁻¹ for the three dose levels. The plasma concentrations of bexagliflozin as a function of time are shown in FIG. 4.

TABLE 4
DEGEE/PEG-400 Formulations

| Ingredient | F16D-E-V2 | F16D-E-V3 | F16D-E |
|---|---|---|---|
| Bexagliflozin (mg) | 3 | 10 | 30 |
| Diethylene glycol | 0.3 | 0.3 | 0.3 |
| ethyl ether (mL) | | | |
| PEG-400 (mL) | 0.2 | 0.2 | 0.2 |
| Fumarate Buffer | 0.5 | 0.5 | 0.5 |
| pH 6.5 (mL) | | | |

TABLE 5
PK Parameters Produced by
DEGEE/PEG-400 Formulation

| Dose (mg kg⁻¹) | 0.3 | 1 | 3 |
|---|---|---|---|
| t_{1/2} (h) | 4.5 | 6.56 | 6.34 |
| Tₘₐₓ (h) | 0.5 | 0.5 | 1.0 |
| Cₘₐₓ (ng mL⁻¹) | 233 | 748 | 846 |
| AUC₀₋ₜ (ng h mL⁻¹) | 459 | 1816 | 3987 |

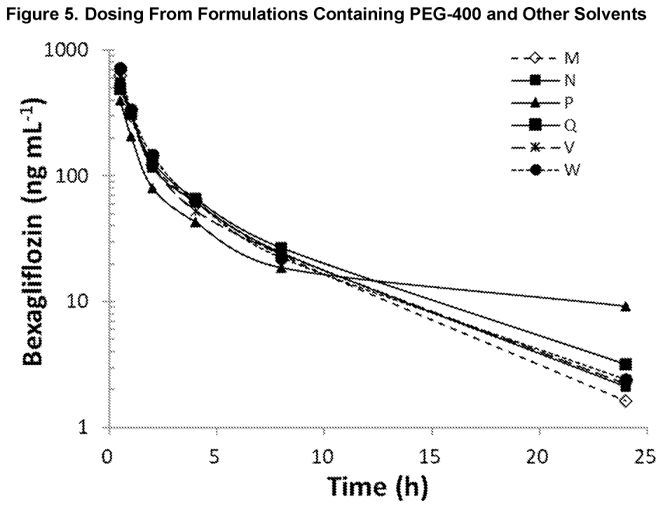

A number of additional liquid formulations were prepared as described in Table 6 and tested in four cats each. Formulation P produced a different profile compared to the others, which were closely similar (FIG. 5). At the lower proportions of DEGEE used, an effect on pharmacokinetics was not seen. The highest absorption (Cₘₐₓ) was produced by formulation W, and resulted in the highest AUC (Table 7). The next highest Cₘₐₓ values were produced by formulations M and V, and the lowest Cₘₐₓ and AUC values were produced by formulation P (Table 7).

TABLE 6
Additional Liquid Formulations

| Formulation Composition | M | N | P | Q | V | W |
|---|---|---|---|---|---|---|
| Bexagliflozin (mg) | 10 | 10 | 10 | 10 | 10 | 10 |
| Propylene Glycol (μL) | | | 150 | | 100 | 150 |
| PEG-400 (μL) | 300 | 300 | 150 | 300 | 300 | 300 |
| Poloysorbate 80 (mg) | — | — | 50 | — | 50 | 50 |
| Sucralose (mg) | — | — | 5 | — | — | — |
| Diethylene glycol ethyl ether | 200 | 100 | 50 | — | 50 | — |
| (μL) | | | | | | |
| 2-Pyrrolidone (μL) | — | — | — | 200 | — | — |
| Citrate buffer (pH 6.5) | QS to 1 | QS to 1 | QS to 1 | QS to 1 | QS to 1 | QS to 1 |
| | mL | mL | mL | mL | mL | mL T |
| Buffer concentration (approx.) | 100 mM | 100 mM | 100 mM | 100 mM | 100 mM | 100 mM |

TABLE 7
PK Parameters for Additional Liquid Formulations

| Formulation | M | N | P | Q | V | W |
|---|---|---|---|---|---|---|
| t_{1/2} (h) | 3.86 | 3.67 | 3.92 | 4.40 | 4.38 | 4.43 |
| Tₘₐₓ (h) | 0.50 | 0.50 | 0.50 | 0.50 | 0.50 | 0.50 |
| Cₘₐₓ | 634.7 | 554.0 | 437.7 | 488.9 | 616.2 | 721.7 |
| (ng mL⁻¹) | | | | | | |
| AUC₀₋ₜ | 1165.7 | 1096.8 | 876.7 | 1096.2 | 1131.0 | 1263.8 |
| (ng h mL⁻¹) | | | | | | |

### Pharmacokinetics Produced by Softgel Formulations

To further characterize the potential utility of DEGEE as an excipient, a flavored softgel delivery system was explored. The compositions were flavored encapsulated liquids prepared with two solvent compositions and two palatants (salmon and tuna flavors). Table 8 and Table 9 present the softgel compositions prepared.

TABLE 8
Softgel Formula B
Lot Quantity 7,500 Softgels

| g per softgel | Item description | Mass per batch | |
|---|---|---|---|
| 0.07660 | Diethylene glycol ethyl ether | 574.5 | g |
| 0.05600 | D-α-tocopherol polyethylene | 420.0 | g |
| | glycol succinate, NF | | |
| 0.00500 | Bexagliflozin | 37.5 | g |
| 0.01600 | Povidone, USP (K-value 90) | 120.0 | g |
| 0.00640 | Palatant | 48.0 | g |
| 0.160 g | Total | 1200.0 | g |

TABLE 9
Softgel Formula D
Lot Quantity 7,500 Softgels

| g per softgel | Item description | Mass per batch | |
|---|---|---|---|
| 0.07660 | Diethylene glycol ethyl ether | 574.5 | g |
| 0.05600 | Polyoxyl 40 Hydrogenated | 420.0 | g |
| | Castor Oil, NF | | |
| 0.00500 | Bexagliflozin | 37.5 | g |
| 0.01600 | Povidone, USP (K-value 90) | 120.0 | g |
| 0.00640 | Palatant | 48.0 | g |
| 0.160 g | Total | 1200.0 | g |

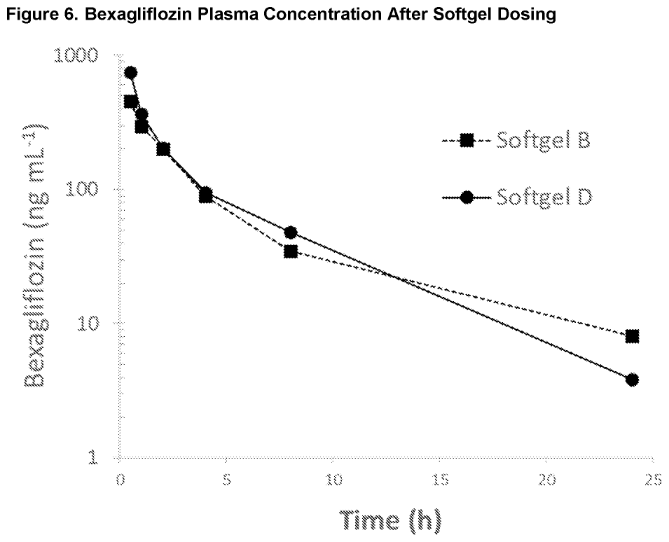

Four laboratory cats were dosed with one softgel each from the two preparations described above. Plasma was collected at pre-dose and at 0.5, 1, 2, 4, 8 and 24 h post-dose and analyzed by an HPLC MS/MS method. FIG. 6 shows that the two formulations produced nearly identical plasma concentrations from 2 h onward. The Cₘₐₓ produced by softgel formula D was greater, 747 ng mL⁻¹ than the Cₘₐₓ for softgel B, 458 ng mL⁻¹, and the AUC₀₋ₜ (AUC₀₋₂₄) values reflected this difference, with formula D producing an AUC₀₋ₜ of 1752 ng h mL⁻¹ and formula B producing 1430 ng h mL⁻¹ (Table 10). The dose level in mg kg⁻¹ was nearly identical, 1.78 for the formula D dosing and 1.79 for the formula B dosing.

TABLE 10
PK Parameters for Softgel Formulations

| Formulation | B | D |
|---|---|---|
| Tₘₐₓ (h) | 0.50 | 0.50 |
| t_{1/2} (h) | 4.91 | 4.21 |
| Cₘₐₓ (ng mL⁻¹) | 458 | 747 |
| AUC₀₋ₜ (ng h mL⁻¹) | 1430 | 1752 |

### Pharmacokinetics Produced by Tablet Formulations

A dose range-finding study was conducted with tablets prepared using a polacrilex resin slurry process. For the evaluation of pharmacodynamics, animals were fed canned food, to promote a constant daily urine volume and proper collection of urine samples. Water was supplied ad libitum.

All animals were administered test article at each dose level. The 2.5 mg dose was delivered as half of one 5 mg tablet cleaved with a pill cutter. The 5 mg dose was delivered as one 5 mg tablet per cat, the 10 mg dose was delivered as one 10 mg tablet per cat and the 30 mg dose was delivered as two 15 mg tablets per cat. For the pharmacokinetics study, test articles were dosed after the sampling of blood at pre-dose (0 h) time point. For the pharmacodynamics study, test articles were administered daily at 9:00-9:30 for four consecutive days and urine excreted by the animals for the last three days (after the dose and before next dose) was collected.

Collections were performed for three days to diminish the consequences of day-to-day variation of urine excretion. The bexagliflozin content per tablet for the 5 mg tablets was significantly lower than expected (3.9 mg, 78% of target content). For convenience in referring to dose levels, the nominal dosages are used in this report, but calculations of dose-response relationships in mg kg⁻¹ were based on the measured bexagliflozin content.

Blood samples were collected from the acral vein at the scheduled time points. Samples of 0.1-0.3 mL were drawn into 1.5 mL tubes containing dipotassium EDTA as an anticoagulant. The samples were immediately mixed by gently inverting the tube approximately 8 times, making sure that the blood on the bottom of the tube had been thoroughly mixed. Immediately following mixing, the tubes were placed on crushed ice and kept chilled until centrifugation. Samples were centrifuged within 1 hour of collection and plasma was harvested. The tubes were spun for 5 min at 4° C. with the centrifuge set at 5,000 rpm. Following centrifugation, the tubes were returned to crushed ice and supernatants transferred into microcentrifuge tubes and frozen. The tubes were stored frozen in a −20° C. freezer at the testing facility prior to shipment for analysis.

Plasma bexagliflozin concentration was measured by an LC-MS/MS method. Separations were performed using an Agilent 1200 equipped with a Thermo Betasil C₁₈ 50×2.1 mm, 5 μm column or a Phenomenex Gemini C₁₈ 50×4.5 mm, 5 μm column. Analyte detection was performed with an ABI 4000 or AB Sciex QTRAP 5500 mass spectrometer.

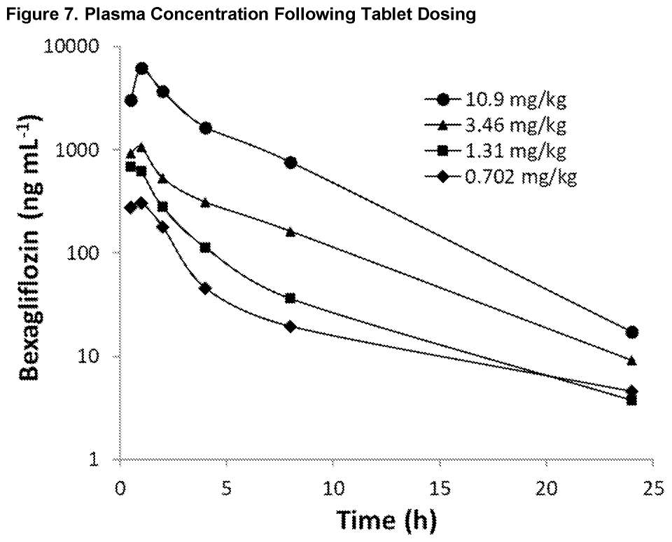

The plasma bexagliflozin concentrations as a function of time are shown in FIG. 7. A dose-dependent increase in exposure was observed with some differences in profile as the dose increased. The contribution of the distribution phase was most pronounced at the lowest dose levels, possibly indicating a saturable compartment or saturable metabolic process. The dose-normalized Cₘₐₓ was somewhat variable and did not show any notable trend with increasing dose level, whereas the dose-normalized AUC was higher for the highest dose level (Table 11).

TABLE 11
PK Parameters Following Tablet Dosing

| Dose (mg kg⁻¹) | 0.70 | 1.31 | 3.46 | 10.9 |
|---|---|---|---|---|
| Cₘₐₓ (ng mL⁻¹) | 341 | 780 | 1220 | 6169 |
| AUC₀₋ₜ (ng h mL⁻¹) | 885 | 1972 | 4679 | 24472 |
| Cₘₐₓ/D (ng mL⁻¹ | 488 | 595 | 353 | 566 |
| per mg kg⁻¹) | | | | |
| AUC₀₋ₜ/D (ng h | 1264 | 1505 | 1352 | 2245 |
| mL⁻¹ per mg kg⁻¹) | | | | |
| t_{1/2} (h) | 2.58 | 3.95 | 3.95 | 2.99 |
| Tₘₐₓ (h) | 1.00 | 0.75 | 1.00 | 1.00 |

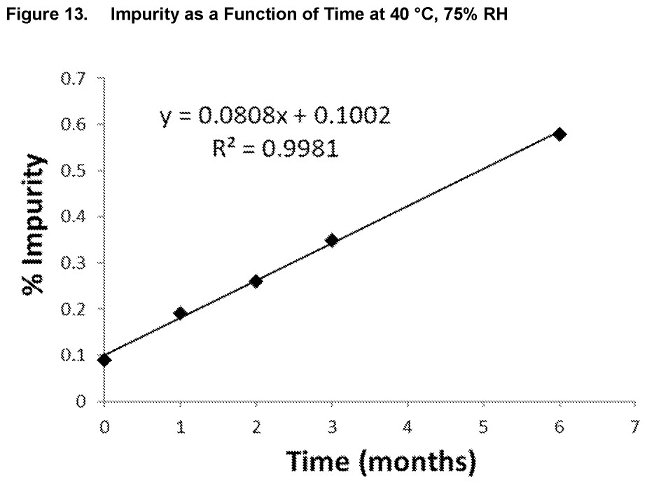

Under high temperature and humidity conditions, the resin slurry formulation accumulated an impurity that was the product of cleavage of the cyclopropyl ether (FIG. 13). It was hypothesized that the ion exchange resin could be responsible for the formation of the impurity.

To evaluate this, three candidate alternate formulations were prepared: A-Na, prepared in the same manner as the acidic resin slurry formulation, but incorporating a neutralization step in which the protons of the resin were replaced with sodium ions; DB-A-H, prepared by dry blend of the resin with the remaining excipients (and leaving the resin in the hydrogen ion form); and DB, a dry blend of the active ingredient with the remaining excipients, excluding the ion exchange resin. The relative pharmacokinetics following a single dose administration to six cats were determined.

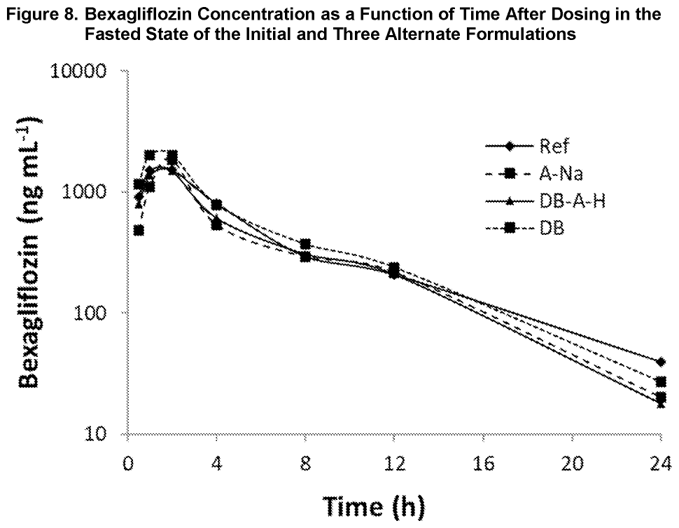

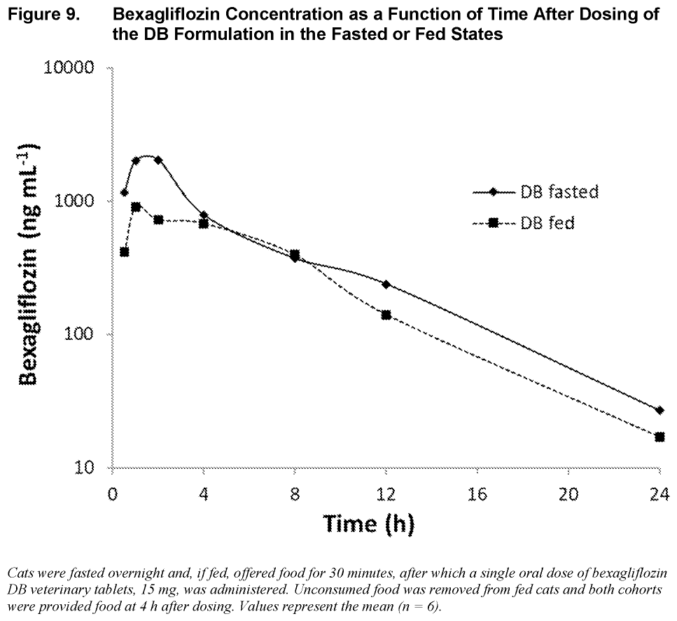

FIG. 8 shows that the plasma concentrations as a function of time were broadly similar among the alternate formulations. Table 12 summarizes the inferred pharmacokinetic parameters. Formulations A-Na and DB-A-H produced somewhat lower Cₘₐₓ and AUC than the acidic resin slurry formulation, whereas formulation DB produced greater exposure by both measures. The consequences of tablet delivery in the fasted or the fed state are presented in FIG. 9.

TABLE 12
Pharmacokinetic Parameters for Bexagliflozin Following Single
Administration of Different Formulations to Cats

| | | Cₘₐₓ | AUC₀₋ₜ | | | CL_{Z}/F | |
|---|---|---|---|---|---|---|---|
| Formu- | Prandial | (ng | (ng h | t_{1/2} | Tₘₐₓ | (L h⁻¹ | V_{Z}/F |
| lation | State | mL⁻¹) | mL⁻¹) | (h) | (h) | kg⁻¹) | (L kg⁻¹) |
| Ref | Fasted | 2377 | 9403 | 5.5 | 2.0 | 0.62 | 4.85 |
| Ref | Fed | 1527 | 8555 | 2.9 | 4.0 | 1.29 | 3.22 |
| A-Na | Fasted | 2157 | 8484 | 3.93 | 2.0 | 0.64 | 3.64 |
| DB-A-H | Fasted | 1928 | 8548 | 3.62 | 1.5 | 0.70 | 3.59 |
| DB | Fasted | 2762 | 11080 | 3.84 | 1.0 | 0.54 | 2.98 |
| DB | Fed | 1532 | 6829 | 3.75 | 3.0 | 0.79 | 4.20 |

Cats in all but the fed cohort were fasted overnight and administered a single dose of bexagliflozin. Fed cats were dosed within 10 min after completion of feeding. Blood samples were drawn at 0.5, 1, 2, 4, 8 and 24 h after dosing. Values represent the mean (n = 6) except Tₘₐₓ (median).

To evaluate the pharmacokinetic consequences of a food reward offered to fasted cats after each dosing session, in two studies fasted cats were dosed with ten tablets of the DB formulation in four sessions of two or three tablets per session. In the first study, cats were not offered a food reward but were returned to their cages after dosing. In the second study, after each session cats were offered a food reward (approximately 5 g of the standard diet). All cats consumed all reward portions.

Table 13 compares the consequences for pharmacokinetic parameters of the administration of ten tablets with or without a food reward. The Cₘₐₓ and AUC values have been divided by 10 to allow the linearity of the pharmacokinetics to be assessed. As Table 13 shows, bexagliflozin exposure by AUC is highly dose-linear and the effect of a food reward does not appear to be substantial. The dose-normalized Cₘₐₓ produced by 10 tablets is about 30% lower than the Cₘₐₓ produced by a single tablet (Table 13).

TABLE 13
Pharmacokinetic Parameters for Bexagliflozin Following Single or
Multiple Administration of Different Formulations to Cats

| | | Cₘₐₓ | AUC₀₋ₜ | | | CL_{Z}/F | V_{Z}/F |
|---|---|---|---|---|---|---|---|
| Formu- | Prandial | (ng | (ng h | t_{1/2} | Tₘₐₓ | (L h⁻¹ | (L |
| lation | State | mL⁻¹) | mL⁻¹) | (h) | (h) | kg⁻¹) | kg⁻¹) |
| Ref | Fasted | 2377 | 9403 | 5.5 | 2.0 | 0.62 | 4.85 |
| DB | Fasted | 2762 | 11080 | 3.84 | 1.0 | 0.54 | 2.98 |
| 10 DB/10 | Fasted | 1949 | 10769 | 10.9 | 2.00 | 0.46 | 7.30 |
| 10 DB/10 | Reward | 1981 | 10116 | 5.17 | 2.00 | 0.50 | 3.73 |

Cats were fasted overnight and administered a single dose or ten doses of bexagliflozin in the indicated formulations. In the reward experiment, cats were offered 5 g of food after each dosing session. In the admix experiments, 10 crushed tablets were added to the indicated masses of food.
Blood samples were drawn at 0.5, 1, 2, 4, 8 and 24 h after dosing of a single tablet. An additional specimen was drawn at 48 h for cats dosed with 10 tablets. Values represent the mean (n = 6) except Tₘₐₓ (median). The Cₘₐₓ and AUC values for cats administered 10 tablets have been divided by 10.

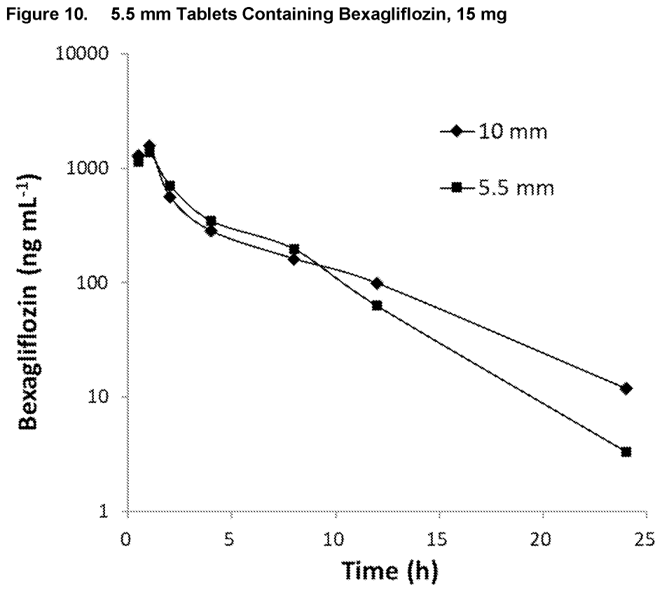

Similar pharmacokinetics could be produced by smaller tablets. A comparison of the plasma concentration profiles produced by 10 mm pentagonal tablets (composition in Table 35) and 5.5 mm round tablets (composition in Table 38) is presented in FIG. 10.

### Pharmacodynamics Produced by Veterinary Formulations

### Pharmacodynamics Produced by Capsule Formulations

The pharmacodynamics of inhibitors of renal glucose reuptake can be easily monitored by measurement of urinary glucose excretion (UGE). The sodium-linked glucose transporters SGLT1 and SGLT2 are expressed in the second and first segments, respectively, (pars recta and pars convoluta) of the renal proximal tubule. There are no substantive sources or sinks for glucose downstream of the proximal tubule and hence net urinary glucose excretion measures the degree to which renal reuptake has been compromised by inhibition of these transporters.

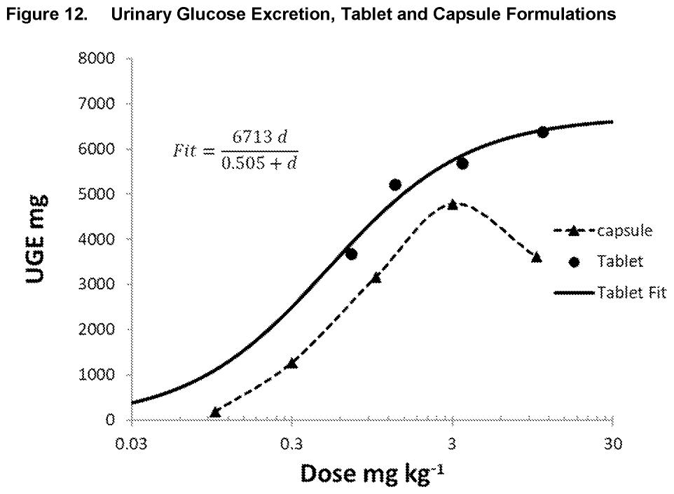

As described above, 4 laboratory cats were administered bexagliflozin as either a blend with microcrystalline cellulose or as pure bexagliflozin, in each case in gelatin capsules. Bexagliflozin induced a significant glucosuria in healthy cats. At 3 mg kg⁻¹, total urinary glucose excreted over a period of 24 hours was 4.78 g (Table 14). The glucosuria was dose dependent, with an ED₅₀ of 0.56 mg kg⁻¹, and appeared to be maximal at 3 mg kg⁻¹ of test article. Glucosuria following test article administration in the fasted state was higher than glucosuria following administration in the fed state (Table 14, bottom row). A graphical depiction of these results can be found in FIG. 12.

TABLE 14
Urinary Glucose Excretion After Capsule Dosing

| Dose (mg kg⁻¹) | 0-8 h | 8-24 h | 0-24 h |
|---|---|---|---|
| 0.1 | 0 ± 0 | 195.5 ± 145.7 | 195.5 ± 145.7 |
| 0.3 | 0 ± 0 | 1276.0 ± 355.5 | 1276.0 ± 355.5 |
| 1 | 357.0 ± 357.0 | 2813.2 ± 502.0 | 3170.2 ± 338.4 |
| 3 | 1414.5 ± 461.8 | 3367.6 ± 440.0 | 4782.1 ± 515.5 |
| 10 | 1370.2 ± 429.7 | 2241.6 ± 1225.2 | 3611.7 ± 1326.2 |
| 3 fed | 189.4 ± 189.4 | 2640.7 ± 766.9 | 2830.1 ± 613.2 |

Values represent the mean ± SD (n = 4) in mg of glucose.

### Pharmacodynamics Produced by Liquid Formulations

The pharmacodynamics produced by liquid formulations of bexagliflozin were also evaluated. Cats were fed daily with Whiskas Cat Food in Can (Ocean Fish) and bexagliflozin was delivered orally daily with different formulations derived from F16D as shown in Table 15.

TABLE 15
DEGEE/Glycerol Formulations

| Ingredient | F16D-V0 | F16D-V1 | F16D-V2 | F16D-V3 | F16D |
|---|---|---|---|---|---|
| Bexagliflozin (mg) | 0 | 1 | 3 | 10 | 30 |
| DEGEE (ml) | 0.3 | 0.3 | 0.3 | 0.3 | 0.3 |
| Glycerol (ml) | 0.2 | 0.2 | 0.2 | 0.2 | 0.2 |
| PBS, pH 7.4 (ml) | 0.5 | 0.5 | 0.5 | 0.5 | 0.5 |

Cats were dosed with F16D-V0 (0.1 mL kg⁻¹) for two days (vehicle control), and urine samples were collected on the second day for glucose excretion analysis. Similarly, cats were dosed daily with F16D-V1 (0.1 mL kg⁻¹) from Day 1 to Day 4 at 0.1 mg kg⁻¹ dose, and urine samples were collected from Day 2 to Day 4 for glucose excretion analysis; cats were dosed daily with F16D-V2 (0.1 mL kg⁻¹) from Day 5 to Day 8 at 0.3 mg kg⁻¹ dose, and urine samples were collected from Day 6 to Day 8 for glucose excretion analysis; cats were dosed daily with F16D-V3 (0.1 mL kg⁻¹) from Day 9 to Day 12 at 1 mg kg⁻¹ dose, and urine samples collected from Day 10 to Day 12 for glucose excretion analysis; and cats were dosed daily with F16D (0.1 mL kg⁻¹) from Day 13 to Day 16 at 3 mg kg⁻¹ dose, and urine samples were collected from Day 14 to Day 16 for glucose excretion analysis.

Urine samples were analyzed using a HITACHI 7600-010 automatic biochemistry analyzer. Urine glucose and creatinine concentrations were measured. Data were uncorrected for possible species and/or analyte source differences in sensitivity and/or measurement response.

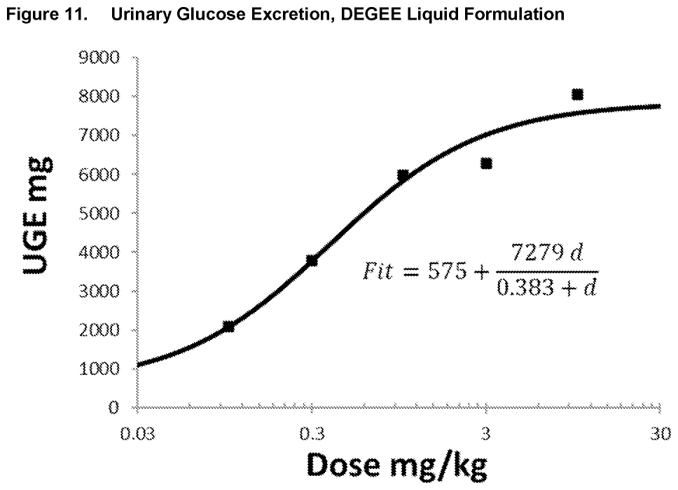

The results are shown in FIG. 11. The maximum daily glucose excretion was predicted to be 7854 mg with a half-maximal dose (ED₅₀) of 0.383 mg kg⁻¹ day⁻¹.

### Pharmacodynamics Produced by Tablet Formulations

The pharmacodynamics elicited by bexagliflozin delivered by tablets was also evaluated. A single dose of bexagliflozin tablets was administered to each of four healthy cats. The tablets were manufactured with consistent size and shape and with a characteristic odor comparable to that of dry food for cats.

Bexagliflozin administered as tablets for veterinary use induced a significant glucosuria in healthy cats. Glucosuria, as indicated by urinary glucose excretion and glucose to creatinine ratio, showed a logistic behavior with an ED₅₀ of 0.505 mg kg⁻¹ (corrected for assay) for UGE as shown in FIG. 12. These dose-response curves did not recapitulate the previously observed decline in glucosuria above the 3 mg kg⁻¹ dose level for capsule administration (FIG. 12).

### Palatability of Veterinary Formulations

Whether an oral medication can be conveniently delivered to an animal depends on the acceptability of the dosage form. Many drugs have what humans consider objectionable tastes and are delivered to humans by the oral route in capsules or film-coated tablets that block direct contact of the active ingredient with the taste receptors of the oral cavity. Animals can be difficult to dose with oral solid dosage forms, and cats are particularly resistant. As a result, favored dosing forms for cats tend to be oral solutions that can be administered to the back of the mouth by a dropper or syringe, provoking a reflexive swallowing that allows the majority of the dosage to be delivered in most cases. However, it is difficult to mask unwanted taste perception.

Taste perception varies by species and within species. Among hypercarnivores, (animals with a diet that is more than 70% flesh), loss of taste receptors for sugars is common (Jiang et al., 2012 Proc Natl Acad Sci USA 109:4956 doi: 10.1073/pnas.1118360109). Cats in particular are known to lack sweet taste perception and have accumulated inactivating mutations in the genes encoding receptors for sweet substances (Li et al. 2006 J Nutr 136:1932S doi: 10.1093/jn/136.7.1932S). The variation in taste perception makes it important to establish the acceptability of candidate oral formulations by palatability testing in the target species.

Palatability testing for bexagliflozin veterinary products was performed for oral solutions and oral tablet dosage forms as described in the following examples.

### Palatability of Liquid Formulations

A palatability study was conducted to evaluate the acceptability of some candidate liquid formulations. The study utilized domestic short hair cats that were between 402 and 932 days of age at the start of dosing. Physiologically, cats were healthy, ranging in body weight from 2.5 to 7.1 kg.

Twenty-five cats (7 neutered male, 18 female) were acclimated to study conditions for seven days. At the end of the acclimation period, 24 (7 neutered male, 17 female) were deemed eligible and entered the dosing period.

Each cat was acclimated to the acceptability testing procedure on Days −3 to −1, prior to starting acceptability testing, with a commercial treat. Each cat was acclimated to the acceptability testing procedure on Days −3 to −1, prior to starting acceptability testing, with a commercial treat. A liquid pet treat was offered to each cat on Days −3 to −1.

A total volume of 0.5 mL was administered to each cat, then the test article or treat was administered orally according to the following general procedure:
The dosing technician ensured that the mouth contained no food or other objects;
The cat's head was tilted backwards with the nose upward;
The dosing syringe was positioned at the back of the mouth and the entire article gently administered.

Test article acceptability was scored as follows:
3: Consumed all of article willingly;
2: Consumed article with minimal restraint, possibly required a brief pause in dosing;
1: Consumed article but only with considerable restraint and possibly required multiple dosing efforts;
0: Unable to administer full dose, cat became excessively fractious, with or without hypersalivation, retching, and loss of article while dosing.

Acceptance was defined as a score of 2 or 3.

Table 16 presents the formulation compositions. The ingredients were bexagliflozin (10 mg mL⁻¹), butylated hydroxyanisole (BHA), diethylene glycol ethyl ether (DEGEE), polyethylene glycol, molecular weight 400 (PEG-400), glycerol, bacon flavor and sodium citrate buffer (100 mM), pH 6.5.

TABLE 16
Effects of Bacon Flavor Palatant on Acceptability

| | | | | PEG- | | | |
|---|---|---|---|---|---|---|---|
| Formu- | Bexagliflozin | BHA | DEGEE | 400 | Glycerol | Palatant | Citrate |
| lation | (g) | (g) | (μL) | (μL) | (μL) | (μL) | Buffer |
| F | 0.1000 | N/A | 3000 | 2000 | N/A | 20 | QS to |
| | | | | | | | 10 mL |
| G | 0.1000 | N/A | 3000 | N/A | 2000 | 20 | QS to |
| | | | | | | | 10 mL |
| H | 0.1003 | 0.01 | 3000 | 2000 | N/A | 20 | QS to |
| | | | | | | | 10 mL |
| J | 0.1000 | N/A | 3000 | 2000 | N/A | 40 | QS to |
| | | | | | | | 10 mL |
| K | 0.1000 | N/A | 3000 | 2000 | N/A | 60 | QS to |
| | | | | | | | 10 mL |
| L | 0.1000 | N/A | 3000 | 2000 | N/A | 100 | QS to |
| | | | | | | | 10 mL |

The scores for dosing are shown in Table 17. Although many cats were scored as 3, meaning they willingly accepted the doses, immediate reactions to consumption of test article formulations included hypersalivation and/or foaming at the mouth, gagging, and squinting of the eyes. Additionally, all formulations were vomited at least once with the exception of formulations H and J, which had the greatest number of scores of 1.

TABLE 17
Individual Scores

| | Scores Formulation | | | | | |
|---|---|---|---|---|---|---|
| Score | F | G | H | J | K | L |
| 0 | 0 | 0 | 0 | 0 | 0 | 0 |
| 1 | 1 | 0 | 4 | 5 | 0 | 3 |
| 2 | 5 | 3 | 6 | 2 | 5 | 4 |
| 3 | 10 | 13 | 6 | 9 | 11 | 9 |
| Total | 16 | 16 | 16 | 16 | 16 | 16 |

Formulations G and K had 100% acceptance (Table 18). Of the remaining formulations, acceptance was ranked as follows: F>L>H. Formulation J was not considered acceptable with an acceptance rate of 69%.

TABLE 18
Acceptance for Bacon Flavor Formulations

| Acceptance | Formulations |
|---|---|
| Formulation | Percent Acceptance |
| F | 94 |
| G | 100 |
| H | 75 |
| J | 69 |
| K | 100 |
| L | 81 |

Another palatability study utilized domestic short hair cats that were between 163 and 970 days of age at the start of dosing. Physiologically, cats were healthy, ranging in body weight from 2.8 to 4.1 kg. Twenty-six cats (7 male, 19 female) were acclimated to study conditions for seven days. At the end of the acclimation period, 24 cats (7 male, 17 female) were deemed eligible and entered the dosing period.

Each cat was acclimated to the acceptability testing procedure on Days −3 to −1, prior to starting acceptability testing, with a commercial treat. A liquid pet treat was offered to each cat on Days −3 to −1. A total volume of 0.5 mL was administered to each cat.

The article or treat was administered orally according to the following general procedure:
The dosing technician ensured that the mouth contained no food or other objects;
The cat's head was tilted backwards with the nose upward;
The dosing syringe was positioned at the back of the mouth and the entire article gently administered.

Test article acceptability was scored as follows:
3: Consumed all of article willingly;
2: Consumed article with minimal restraint, possibly required a brief pause in dosing;
1: Consumed article but only with considerable restraint and possibly required multiple dosing efforts;
0: Unable to administer full dose, cat became excessively fractious, with or without hypersalivation, retching, and loss of article while dosing.
Acceptance was defined as a score of 2 or 3.

Table 19 presents the formulation compositions. The ingredients were bexagliflozin (10 mg mL⁻¹), propylene glycol (PG), polyethylene glycol, molecular weight 400 (PEG-400), polyoxyethylene (20) sorbitan monooleate (PS 80), diethylene glycol ethyl ether (DEGEE), isosorbide dimethyl ether (IDE), propylene glycol monolaurate (PG-12), 2-pyrrolidinone and citrate buffer (100 mM), pH 6.5.

TABLE 19
Formulation Compositions

| | | | PEG- | PS | | | PG | | Citrate |
|---|---|---|---|---|---|---|---|---|---|
| | Bexa | PG | 400 | 80 | DEGEE | IDE | 12 | Pyrrolidone | Buffer |
| Formulation | (g) | (μL) | (μL) | (μL) | (μL) | (μL) | (μL) | (μL) | (mL) |
| AH | 0.1004 | 1500 | 5000 | N/A | N/A | N/A | N/A | N/A | QS to 10 |
| | | | | | | | | | mL |
| AJ | 0.1004 | 1500 | 5000 | 200 | N/A | N/A | N/A | N/A | QS to 10 |
| | | | | | | | | | mL |
| Q | 0.1004 | N/A | 3000 | N/A | N/A | N/A | N/A | 2000 | QS to 10 |
| | | | | | | | | | mL |
| AK | 0.1002 | 1500 | 5000 | N/A | 500 | N/A | N/A | N/A | QS to 10 |
| | | | | | | | | | mL |
| AL | 0.1001 | 1500 | 5000 | N/A | N/A | 1000 | N/A | N/A | QS to 10 |
| | | | | | | | | | mL |
| AM | 0.1001 | 1500 | 5000 | N/A | N/A | N/A | 1000 | N/A | QS to 10 |
| | | | | | | | | | mL |

The scores for dosing are shown in Table 20. Post-dosing vomiting was noted sporadically. Each formulation was vomited a single time post-dosing with the exception of formulation AH, which was poorly tolerated.

TABLE 20
Individual Scores

| | Formulation | | | | | |
|---|---|---|---|---|---|---|
| Score | AH | AJ | Q | AK | AL | AM |
| 0 | 0 | 0 | 0 | 0 | 0 | 0 |
| 1 | 11 | 4 | 4 | 1 | 8 | 3 |
| 2 | 1 | 5 | 1 | 1 | 2 | 3 |
| 3 | 4 | 7 | 11 | 14 | 6 | 10 |
| Total | 16 | 16 | 16 | 16 | 16 | 16 |

Formulations AJ, AK, AM, and Q were considered acceptable, all having acceptance rates greater than 70%. Formulations AL and AH were not considered acceptable with acceptance rates of 50 and 31%, respectively. Formulation AK, consisting of 50% PEG-400, 15% propylene glycol and 5% diethylene glycol ethyl ether, was the most readily accepted and produced the least hypersalivation (Table 21).

TABLE 21
Acceptance Scores and Hypersalivation

| Formu- | Score | Times | % | Hyper- |
|---|---|---|---|---|
| lation | 2 or 3 | dosed | Acceptance | salivation |
| AH | 5 | 16 | 31 | 11 |
| AJ | 12 | 16 | 75 | 4 |
| AK | 15 | 16 | 94 | 1 |
| AL | 8 | 16 | 50 | 8 |
| AM | 13 | 16 | 81 | 3 |
| Q | 12 | 16 | 75 | 4 |

Oral solutions containing bexagliflozin were also evaluated in an experiment involving 28 mature, domestic short hair cats, 19 to 33 months of age and weighing between 2.40 to 6.90 kg, served as candidates for the study. All cats were judged to be in good general health, as confirmed by physical examination and daily general health observations. Each cat originated from the facility's resident colony, and was permanently identified with a unique tattoo number inscribed in the left pinna. Demographic information for the enrolled cats is presented in Table 22.

TABLE 22
Group Assignments, Sex and Body Mass

| Formulation | Cat I.D. | Sex | Mass (kg) |
|---|---|---|---|
| A | 15KMQ3 | F | 3.05 |
| A | 15KMH2 | F | 2.65 |
| A | 15ENE5 | F | 3.95 |
| A | 14JMV1 | MC | 6.80 |
| B | 15ENA5 | F | 2.50 |
| B | 15EMH4 | F | 3.05 |
| B | 15KMS3 | F | 3.70 |
| B | 15EDR2 | MC | 6.90 |
| C | 14DLH2 | MC | 5.70 |
| C | 15EMS6 | F | 2.70 |
| C | 15ENI5 | F | 3.40 |
| C | 15KLX4 | F | 2.95 |
| D | 15KMI3 | F | 3.60 |
| D | 14JOQ1 | MC | 5.55 |
| D | 15ELO3 | F | 2.75 |
| D | 15EKH5 | F | 3.00 |
| E | 15KMG4 | F | 2.40 |
| E | 15ENI3 | F | 3.55 |
| E | 15KNJ3 | F | 4.10 |
| E | 15EKH8 | F | 3.00 |
| F | 14DLA2 | MC | 6.25 |
| F | 15ENI4 | F | 3.20 |
| F | 15ELT2 | F | 2.95 |
| F | 15EMM2 | F | 2.90 |

MC = male castrate;
F = intact female

Cats were eligible for inclusion if they were mature (>6 months of age), judged to be healthy on the basis of physical examinations and daily general health observations, and had a temperament conducive to experimental procedures. Females could not be pregnant or lactating. Candidates could also be excluded if they proved to be particularly resistant to oral administration during the placebo training sessions. Candidates were acclimated to study conditions for seven days prior to the initial treatment. During the acclimation phase, management, feed, and water were identical to conditions during the treatment phase. General health observations were conducted once daily during the acclimation period.

Also during this interval, three flavored, oral formulations were administered on multiple days to acclimate cats to oral dosing. All candidates received the same training formulation on a given day.

Individual doses of bexagliflozin solution were prepared for each enrolled cat on the basis of its respective group assignment, and an intended standard dose of 0.5 mL. All 24 cats were treated with their assigned formulation once daily on four consecutive days (Days 0 thru 3). The compositions of the oral solutions are shown in Table 23.

TABLE 23
DEGEE Formulations, 10 mg mL⁻¹

| Formulation | | | | | | |
|---|---|---|---|---|---|---|
| Composition | A | B | C | D | E | F |
| Bexagliflozin | 10 | 10 | 10 | 10 | 10 | 10 |
| (mg) | | | | | | |
| PEG-400 (μL) | 200 | — | — | 200 | 200 | 200 |
| DEGEE (μL) | 300 | 300 | 300 | 300 | 300 | 300 |
| Glycerol (μL) | — | 200 | 200 | — | — | — |
| BHA (mg) | — | — | — | — | 1 | — |
| Bacon flavor | | | | 2 | | 2 |
| (μL) | | | | | | |
| Citrate buffer | — | — | QS to 1 | — | — | QS to 1 |
| (pH 6.5) | | | mL | | | mL |
| Fumarate buffer | QS to 1 | QS to 1 | — | QS to 1 | QS to 1 | — |
| (pH 6.5) | mL | mL | | mL | mL | |
| Buffer | 100 | 100 | 100 | 100 | 100 | 100 |
| concentration | mM | mM | mM | mM | mM | mM |
| (approx.) | | | | | | |

DEGEE: diethylene glycol ethyl ether,
BHA: butylated hydroxyanisole.
Buffers had sodium counterions..

Acceptance was based on a binary scoring scheme shown in Table 24.

TABLE 24
Acceptance Scoring Criteria

| Score Designation | Description |
|---|---|
| 0 (Non- | Repeated negative reactions, such |
| acceptance) | as pawing at the mouth, excessive |
| | salivation, retching, vomiting, |
| | spitting, drooling or gagging |
| 1 (Acceptance) | Acceptance of the investigational |
| | veterinary product without |
| | displaying negative reactions above |

Acceptance scores for each formulation are shown in Table 25. Total acceptance scores for the entire study period were summarized as proportions and also expressed as percentages (Table 26).

TABLE 25
Proportion of Positive Acceptance Scores

| Formulation | Day 0 | Day 1 | Day 2 | Day 3 |
|---|---|---|---|---|
| A | 0/4 | 0/4 | 0/4 | 1/4 |
| B | 0/4 | 1/4 | 1/4 | 3/4 |
| C | 3/4 | 1/4 | 2/4 | 2/4 |
| D | 0/4 | 1/4 | 1/4 | 1/4 |
| E | 1/4 | 1/4 | 1/4 | 2/4 |
| F | 2/4 | 3/4 | 2/4 | 3/4 |

TABLE 26
Total Scores and Percentage

| Formulation | Total | Percentage |
|---|---|---|
| A | 1/16 | 6.25 |
| B | 5/16 | 31.25 |
| C | 8/16 | 50.00 |
| D | 3/16 | 18.75 |
| E | 5/16 | 31.25 |
| F | 10/16 | 62.50 |

Formulations A, B, D, and E were acceptable to fewer than 50% of cats treated with the investigational product over the course of the trial. Formulations C and F were acceptable to 50% and 62.5%, respectively, of cats treated with the product.

A further study was conducted to evaluate the acceptability of a number of candidate excipient compositions. In this study only the vehicle was evaluated.

Twenty-four, mature, domestic short hair cats were acclimated to study conditions for three days. Candidate cats were judged to be in good general health, as confirmed by historical health records and daily general health observations.

Each cat originated from the facility's resident colony, and was permanently identified with a unique tattoo number inscribed in the left pinna. Demographic information for enrolled cats is presented in Table 27.

TABLE 27
Group Assignments, Sex and Body Mass

| Formulation | Cat I.D. | Sex | Mass (kg) |
|---|---|---|---|
| M | 14JOQ1 | MC | 5.50 |
| M | 15ELT2 | F | 2.90 |
| M | 15EMM2 | F | 2.95 |
| M | 15KMI3 | F | 3.5 |
| N | 14DLH2 | MC | 5.60 |
| N | 15EKH5 | F | 3.05 |
| N | 15EMS6 | F | 2.75 |
| N | 15KLX4 | F | 4.00 |
| O | 15EDR2 | MC | 7.05 |
| O | 15ENE5 | F | 3.00 |
| O | 15ENI5 | F | 3.60 |
| O | 15KMH2 | F | 2.60 |
| P | 14JMV1 | MC | 6.70 |
| P | 15EMH4 | F | 3.15 |
| P | 15KMG4 | F | 2.50 |
| P | 15KMS3 | F | 3.95 |
| Q | 14DLA2 | MC | 6.40 |
| Q | 15EKH8 | F | 3.05 |
| Q | 15ELO3 | F | 2.75 |
| Q | 15ENI4 | F | 3.20 |
| S | 15ENA5 | F | 2.60 |
| S | 15ENI3 | F | 3.45 |
| S | 15KMQ3 | F | 2.90 |
| S | 15KNJ3 | F | 4.35 |

MC = male castrate;
F = intact female

Table 28 presents the vehicle compositions. The ingredients were propylene glycol, polyethylene glycol, molecular weight 400 (PEG-400), polyoxyethylene sorbitan monooleate (Polysorbate 80), sucralose, diethylene glycol ethyl ether (DEGEE), 2-pyrrolidinone and citrate buffer (100 mM), pH 6.5.

TABLE 28
Vehicle Compositions

| Formulation Composition | M-PBO | N-PBO | O-PBO | P-PBO | Q-PBO | S-PBO |
|---|---|---|---|---|---|---|
| Propylene Glycol (μL) | | | | 150 | | 150 |
| PEG-400 (μL) | 300 | 300 | 300 | 150 | 300 | 200 |
| Polysorbate 80 (mg) | — | — | — | 50 | — | — |
| Sucralose (mg) | — | — | — | 5 | — | — |
| Diethylene glycol ethyl ether | 200 | 100 | 50 | 50 | — | — |
| (μL) | | | | | | |
| 2-pyrrolidone (μL) | — | — | — | — | 200 | — |
| Citrate buffer (pH 6.5) | QS to 1 | QS to 1 | QS to 1 | QS to 1 | QS to 1 | QS to 1 |
| | mL T | mL T | mL T | mL | mL | mL |
| Buffer concentration (approx.) | 100 mM | 100 mM | 100 mM | 100 mM | 100 mM | 100 mM |

Acceptance was based on a binary scoring scheme as previously shown in Table 24.

Acceptance scores for each vehicle are shown in Table 29. Total acceptance scores for the entire study period were summarized as proportions and also expressed as percentages (Table 30).

TABLE 29
Proportion of Positive Acceptance Scores

| Formulation | Day 0 | Day 1 | Day 2 | Day 3 |
|---|---|---|---|---|
| M | 3/4 | 3/4 | 3/4 | 3/4 |
| N | 2/4 | 2/4 | 3/4 | 3/4 |
| O | 3/4 | 4/4 | 4/4 | 4/4 |
| P | 2/4 | 1/4 | 2/4 | 2/4 |
| Q | 3/4 | 4/4 | 4/4 | 3/4 |
| S | 4/4 | 4/4 | 4/4 | 4/4 |

TABLE 30
Total Scores and Percentage

| Formulation | Total | Percentage |
|---|---|---|
| M | 12/16 | 75% |
| N | 10/16 | 62.5% |
| O | 15/16 | 93.75% |
| P | 7/16 | 43.75% |
| Q | 14/16 | 87.5% |
| S | 16/16 | 100% |

Formulations M, O, Q and S were acceptable to ≥75% of cats treated with the respective test article over the course of the trial. Formulations S and O were acceptable to 100% and 93.75% of treated cats, respectively.

### Palatability of Tablet Formulations

To evaluate the palatability of bexagliflozin veterinary tablets, 10 cats were acclimated to study conditions for seven days, during which they were subjected to physical examinations, body weight measurements, acceptability testing training and scoring, and daily clinical observations.

At the end of the acclimation period, eight cats were selected to enter the dosing phase based on signalment characteristics, health, acceptability testing training scores, and amenability to study procedures.

Acceptance was scored during both training and test periods as follows:
3: Consumed all of article from dish
2: Consumed all of article from hand
1: Consumed partial article
0: Did not consume article

Each cat was acclimated to the acceptability testing procedure on Days −3 to −1, prior to starting acceptability testing, with a commercial treat as follows:
A pet treat was offered to each cat at 11:30 (±30 min), after a brief fasting period of at least 2.5 hours, according to the following general procedure:
Cats (while in their cages) were dosed by offering article or treat from a clean, empty food dish;
if the cat removed the article or treat from the dish and did not consume it, it was replaced in the dish;
the time allotted to consume the article or treat was 3 minutes and if it was not consumed in the allotted time it was offered by a gloved hand;
if, after a total offering time of 5 minutes (bowl and hand), the cat did not take the article or treat, the test ended.

Acceptance rates for test article and control article (milbemycin oxime/praziquantel tablets) were 75% and 41% respectively (Table 31). Therefore, the test article was considered acceptable.

TABLE 31
Acceptance Testing Scores

| Score | Control Article | Test Article |
|---|---|---|
| 0: Did not consume article | 12 | 7 |
| 1: Consumed partial article | 7 | 1 |
| 2: Consumed all of article from hand | 0 | 0 |
| 3: Consumed all of article from dish | 13 | 24 |

In a target animal safety study, bexagliflozin veterinary tablets, 15 mg, with the composition shown in Table 35 were delivered by mouth to purpose-bred cats for 182 days. Dosing was adjusted to ensure that the minimum dose was either 5, 15 or 25 mg kg⁻¹ day⁻¹. For male animals in the ≥25 mg kg⁻¹ day⁻¹ cohort as many as 9 tablets per day were administered, in dosing sessions delivering up to 4 tablets per session, separated by a 15 minute rest period between sessions. No major difficulties were encountered and all animals completed the study.

In a field evaluation study, bexagliflozin veterinary tablets, 15 mg, were delivered by mouth to dogs of different breeds for variable lengths of time. Bexagliflozin veterinary tablets, 15 mg, with the composition shown in Table 35 were well accepted and dosing failures were not reported.

In one instance an owner with both a cat and a dog in the household reported that the dog had chewed open the container containing tablets intended for treatment of the cat and consumed all the tablets. No obvious ill effects were observed. Bexagliflozin veterinary tablets, 15 mg, with the composition shown in Table 35 were considered to be attractive to at least one dog.

### Stability of Veterinary Formulations

### Stability of Liquid Formulations

To understand the solvating power of various excipient mixtures and the stability of the resulting solutions after prolonged incubation at room temperature, the formulations presented in were evaluated. Formulation T-PG was found to form crystals after 72 h of incubation but formulations F02A and F02B formed clear solutions that were stable to prolonged room temperature incubation (Table 32). F02A had lower viscosity than F02B (Table 32).

TABLE 32
Solution Stability

| Condition | Duration | T-PG | F02A | F02B |
|---|---|---|---|---|
| Initial | N/A | API completely | API completely | API completely |
| | | dissolved after | dissolved after | dissolved after |
| | | 3 minutes | 8 minutes | 8 minutes at |
| | | at 80° C. | at 40° C. | 40° C. |
| RT | 0 h | Clear solution | Clear solution | Clear solution |
| RT | 24 h | Clear solution | Clear solution | Clear solution |
| RT | 72 h | Precipitated | Clear solution | Clear solution |
| | | (crystals | | |
| | | observed) | | |

| | | | |
|---|---|---|---|
| Viscosity (cP) | 7.91* | 19.2 | 31.5 |
| (Brookfield | | | |
| Rheometer, | | | |
| 25° C.) | | | |

N/A: not applicable. Viscosity was tested on samples after 72 h at RT.
*The sample was reheated to 80° C. to dissolve crystals and cooled to 25° C. for viscosity testing.

The samples were stored at −20° C. for 24 hours followed by thawing at room temperature for 1 hour then stored at 50° C. for 24 hours. The cooling/heating cycle was repeated for a total of three cycles. Viscosity was measured at the end of cycle 3. Solution T-PG did not perform well in this test (Table 33).

TABLE 33
Stability to Freeze/Thaw Cycles

| Condition | Duration | T-PG | F02A | F02B |
|---|---|---|---|---|
| Initial | 0 h | Clear solution | Clear solution | Clear solution |
| Cycle 1: −20° C. | 24 h | Precipitated | Clear solution | Clear solution |
| | | after 2 h* | | |
| | | at −20 ° C. | | |
| Cycle 1: 50° C. | 24 h | Clear solution | Clear solution | Clear solution |
| Cycle 2: −20° C. | 24 h | Precipitated | Clear solution | Clear solution |
| Cycle 2: 50° C. | 24 h | Clear solution | Clear solution | Clear solution |
| Cycle 3: −20° C. | 24 h | Precipitated | Clear solution | Clear solution |
| Cycle 3: 50° C. | 24 h | Clear solution | Clear solution | Clear solution |
| | | (precipitated | (still clear | (still clear |
| | | when left | when left | when left |
| | | at RT | at RT | at RT |
| | | overnight) | overnight) | overnight) |
| Viscosity (cP) | | 6.48 ** | 17.3 | 31.5 |
| (Brookfield | | | | |
| Rheometer, | | | | |
| 25° C.) | | | | |

*When thawed to RT, crystals re-dissolved with shaking.
**The sample was reheated to 80° C. to dissolve crystals and cooled to 25° C. for viscosity testing.

### Stability of Tablet Formulations

The stability of tablet formulations was also explored. The acidic resin slurry tablet formulation exhibited superior palatability and good bioavailability but was discovered to accumulate an impurity in stability testing involving prolonged exposure to storage conditions of 40° C., 75% relative humidity. A linear increase in an impurity resulting from cleavage of the cyclopropyl ether of bexagliflozin was observed (FIG. 13).

The impurity was suspected to be due to the presence of the acidic resin and several formulations were prepared in which the resin was altered, mixed with the excipients in a different manner, or omitted.

The resin-free dry blend tablet with the composition provided in Table 35 was selected for further characterization. Tablets were pressed and bottled in 30 or 90 count high density polyethylene bottles with a child-resistant closure. Representative samples of the bottling run were incubated at 25±2° C. and 60±5% relative humidity for 36 months. The impurity resulting from cleavage of the cyclopropyl ether was present at 0.06%, 0.14% and 0.07% in tablets packaged in 30 count bottles and 0.05%, 0.14% and 0.07% in tablets packaged in 90 count bottles. The assay was 98.5%, 99.6% and 99.8% for tablets in 30 count bottles and 99.6%, 99.7% and 100.2% for tablets in 90 count bottles. It was concluded that the dry blend tablets showed excellent stability characteristics.

### Preparation of Veterinary Formulations

### Preparation of Liquid Formulations

The preparation of the formulations varied by type. Liquid formulations were generally prepared at the experimental site. Some formulations were prepared elsewhere and delivered to the site. The volume of solution prepared was typically 10 mL.

### Preparation of Softgel Formulations

Softgel formulations containing water-soluble vitamin E (D-α-tocopherol polyethylene glycol succinate) were prepared as follows. A stainless steel vessel was charged with D-α-tocopherol polyethylene glycol succinate, NF (420.0 g). The D-α-tocopherol polyethylene glycol succinate was heated until completely melted, not allowing the temperature to exceed 65° C. A stainless steel vessel was charged with diethylene glycol ethyl ether (574.5 g). Under constant mixing the diethylene glycol ethyl ether was heated to 45-55° C., the melted D-α-tocopheryl polyethylene glycol is added, and bexagliflozin (37.5 g) was slowly added and mixed until it had completely dissolved. Then with constant mixing and maintaining the temperature at 45-55° C., povidone, USP (K-value 90), 120.0 g was added and mixed until the composition was homogeneous (10 to 15 min). The temperature was reduced to 40-50° C. and with constant mixing, 48 g of palatant was added and mixing continued until the material was uniform (5 to 10 min). The composition was then de-aerated for not less than 5 min, blanketed with nitrogen and sealed.

Softgel formulations containing polyoxyl castor oil were prepared as follows. A stainless steel vessel was charged with polyoxyl 40 hydrogenated castor oil, NF (420.0 g) and heated until the material was completely melted, keeping the temperature at or below 45° C. A stainless steel vessel was charged with diethylene glycol ethyl ether (574.5 g) and heated to 45-55° C. with mixing. With constant mixing, the melted polyoxyl 40 hydrogenated castor oil was added and followed by slow addition of bexagliflozin (37.5 g), and the composition further mixed until the bexagliflozin had completely dissolved. Then 120.0 g of povidone, USP, (K-value 90) are slowly added and the composition mixed until homogeneous (15 to 30 min). The temperature was reduced to 40-50° C. and with continued mixing 48.0 g palatant was added and mixing continued until the material was uniform (5 to 10 min), then de-aerated for not less than 5 minutes, blanketed with nitrogen and sealed.

### Preparation of Tablet Formulations

An initial resin-based tablet formulation was created by forming a slurry of bexagliflozin and polacrilex resin, removing the excess water from the slurry by centrifugation, drying the centrifuged solids in a fluidized bed dryer and milling the product to produce a powder that could be combined with additional excipients to produce a blend for tableting.

The composition per tablet produced by the resin slurry process is provided in Table 34.

TABLE 34
Composition of Bexagliflozin Tablets, Resin Process

| Component | Quantity | | % |
|---|---|---|---|
| Bexagliflozin | 15 | mg | 4.0 |
| Amberlite IRP64 Polacrilex Resin | 60 | mg | 16.0 |
| Lactose Monohydrate (Flowlac 100) | 48 | mg | 20.0 |
| Microcrystalline Cellulose (Heweten 102) | 154.5 | mg | 41.2 |
| Palatant | 37.5 | mg | 10.0 |
| Pregelatinized Starch (Starch 1500) | 24 | mg | 6.4 |
| Colloidal Silicon Dioxide (Aerosil 200) | 6 | mg | 1.6% |
| Magnesium Stearate (Hyqual) | 3 | mg | 0.8% |
| Total | 375 | mg | 100% |

The composition of a 1 kg batch of bexagliflozin veterinary tablets, 15 mg, produced by a dry blend process is provided in Table 35

TABLE 35
Composition of Bexagliflozin Tablets, Dry Blend

| | Mass per | Mass per | |
|---|---|---|---|
| Ingredient | tablet (mg) | batch (g) | % |
| Bexagliflozin | 15.0 | 40.0 | 4.0% |
| Lactose monohydrate | 135.0 | 360.0 | 36.0% |
| Microcrystalline | 154.5 | 412.0 | 41.2% |
| cellulose | | | |
| Palatant^{a} | 37.5 | 100.0 | 10.0% |
| Pregelatinized starch | 24.0 | 64.0 | 6.4% |
| Colloidal silicon | 6.0 | 16.0 | 1.6% |
| dioxide | | | |
| Magnesium stearate | 3.0 | 8.0 | 0.8% |
| Total | 375.0 | 1000.0 | 100.0% |

^{a}Hydrolyzed chicken liver preparation

The manufacturing of a 1 kg tablet batch was conducted as follows:
Step 1 The bexagliflozin and about 160 g of lactose monohydrate 100 was screened through a #30 mesh hand screen.
Step 2 The colloidal silicon dioxide and palatant was tumble blended in a suitable polyethylene bag for 2 min.
Step 3 The microcrystalline cellulose, half of the pregelatinized starch and the remaining lactose monohydrate were screened through a #30 mesh screen.
Step 4 Step 3 and Step 1 were added to a 4 Quart V-blender shell in the following order:
   (a) half of the product of step 3
   (b) the product of step 1
   (c) half of the product of step 3
Step 5 The remaining half of the pregelatinized starch and magnesium stearate was tumble blended in a suitable sized polyethylene bag for 1 minute then manually screened through a #30 screen using a polypropylene hand scraper.
Step 6 The screened material from Step 5 was added to the 4 Quart V-blender and mixed for 2 minutes at 25 RPM.
Step 7 The blend was discharged into a suitably sized polyethylene bag.

The manufacturing steps for a 75 kg tablet batch are as follows:
Step 1 The following were added in the order listed to a 10 cu. ft. V-blender:
   (a) microcrystalline cellulose, 15.45 kg
   (b) lactose monohydrate, 13.5 kg
Step 2 The ingredients were mixed in the V-blender for 3 minutes at 20 (19-21) RPM.
Step 3 The following were added in the order listed to the same V-blender:
   (a) bexagliflozin, 3.0 kg
   (b) pregelatinized starch, 4.8 kg
   (c) colloidal silicon dioxide, 1.2 kg
   (d) microcrystalline cellulose, 15.45 kg
   (e) lactose monohydrate, 6.75 kg
Step 4 The contents of the V-blender were mixed for 10 minutes at 20 (19-21) RPM without I-bar.
Step 5 The blend was discharged into containers double-lined with PE bags.
Step 6 The blend was milled using a Quadro Comil equipped with a 2A018R01530 or 2C018R01530 (457 micron, 40 mesh equivalent) screen and round impeller at a mill speed of 1200 rpm. The milled material was collected into containers double-lined with PE bags.
Step 7 The following ingredients were milled using a Quadro Comil equipped with a 2A018R01530 or 2C018R01530 (457 micron, 40 mesh equivalent) screen and round impeller at a mill speed of 1200 rpm:
   (a) palatant, 7.5 kg
   (b) lactose monohydrate, 6.75 kg
Step 8 The milled material was collected into containers double-lined with PE bags.
Step 9 The V-blender from step 5 was charged with:
   (a) milled blend from step 7
   (b) milled palatant/lactose monohydrate from step 7
Step 10 The V-blender contents were mixed for 13 minutes at 20 (19-21) RPM.
Step 11 Magnesium stearate (0.6 kg) was screened through a number 30 mesh hand screen and collected in a container double-lined with PE bags.
Step 12 The screened magnesium stearate was added into the V-blender from step 9.
Step 13 The V-blender contents were mixed for 4 minutes at 20 (19-21) RPM.
Step 14 The blend was discharged into tared product containers double-lined with PE bags.
   To produce tablets from the above a tablet press was set up for tablet weight operating limits 375 mg±3%, acceptance limits 375 mg±5%, hardness 7 kp (range 5-10 kp), thickness 5.1 mm (±3 mm), friability not more than 1%.
   Compression was carried out at 20-40 rpm to a filling depth of 8.64 mm with a pre-compression thickness of 2.60 mm and a main compression thickness of 1.78 mm, upper punch pre-penetration 3.5 mm, main penetration 3.5 mm, cam size 4-11 mm.

Preparation of smaller tablets containing 15 mg bexagliflozin was also evaluated. These included both circular and pentagonal tablets.

Three batches of tablets were made using 7 mm pentagonal die tooling. The formulation compositions are presented in Table 36 and the characteristics of tablets are presented in Table 37.

TABLE 36
7 mm Tablet Compositions (mg/unit)

| | DB-003- | DB-004-190304 | DB-006- |
|---|---|---|---|
| Ingredient | 190314 | DB-004-190321 | 190305 |
| Bexagliflozin | 15.0 | 15.0 | 15.0 |
| Lactose Monohydrate | 45.0 | 45.0 | 45.0 |
| Microcrystalline Cellulose | 51.5 | 51.5 | 51.5 |
| Pregelatinized Starch | 8.0 | 8.0 | 8.0 |
| Colloidal Silicon Dioxide | 2.0 | 2.0 | 2.0 |
| Magnesium Stearate | 1.0 | 1.0 | 1.0 |
| Palatant A | 12.5 | 4 | — |
| Palatant B | — | — | 6.5 |
| Total (mg/unit) | 135.0 | 126.5 | 129.0 |

Table 37 shows that the blend uniformity (RSD <3%) and tablet weight variation (<5%) of the tablets met the product requirements.

TABLE 37
7 mm Tablet Characteristics

| | DB-003- | DB-004- | DB-004- | DB-006- |
|---|---|---|---|---|
| Characteristic | 190314 | 190304 | 190321 | 190305 |
| Blend uniformity | 98.1-103.9 | 101.7-104.9 | 98.9-103.8 | 98.3-102.8 |
| (range) | | | | |
| Blend uniformity | 101.2 ± 1.5 | 103.4 ± 1.1 | 101.2 ± 1.4 | 100.7 ± 1.4 |
| (Average ± | | | | |
| SD, %) | | | | |
| Hardness (kp) | 4.5 | 4.5 | 4.9 | 5.0 |
| Thickness (mm) | 3.42 | 3.40 | 3.24 | 3.56 |
| Disintegration | 30 s | 30 s | 44 s | 65 s |
| time | | | | |
| Weight variation | +5% | +5% | +5% | +5% |
| range | | | | |

Table 38 presents the composition of the batches of 5 mm round tablets and 5.5 mm round tablets that produced the results shown in Table 39 and FIG. 10, respectively.

Table 39 presents the characteristics of the 5 mm round tablets. These tablets conformed to the release specifications. The close similarity of the pharmacokinetic profiles shown in FIG. 10 indicate that, within the dry blend formulation, lactose monohydrate can make up 29.6% to 36% of the formulation, microcrystalline cellulose can vary between 33.6% and 41%, pregelatinized starch can be between 5.3% and 6.4% of the total and the palatant can vary between 7.9% and 10.0%, without greatly affecting the in vivo properties imparted by the formulation. For these excipients an acceptable approximate range is 80% to 100% of the relative proportion found in the 10 mm tablets. The glidant silicon dioxide and the lubricant magnesium stearate, which are present in relatively small amounts per tablet, can vary over somewhat larger relative range without greatly affecting the pharmacokinetics. The proportions of silicon dioxide and magnesium stearate are higher in the smaller tablet, 2.6% instead of 1.6% for silicon dioxide and 1.3% instead of 0.8% for magnesium stearate.

TABLE 38
Composition of 5 and 5.5 mm Round Tablets

| Ingredient | mg/unit | % |
|---|---|---|
| Bexagliflozin | 15 | 19.7% |
| Lactose Monohydrate | 22.5 | 29.6% |
| Microcrystalline Cellulose | 25.5 | 33.6% |
| Pregelatinized Starch | 4 | 5.3% |
| Colloidal Silicon Dioxide | 2 | 2.6% |
| Magnesium Stearate | 1 | 1.3% |
| Palatant | 6 | 7.9% |
| Total | 76 | 100.0% |

TABLE 39
Characteristics of 5 mm Tablets

| Criterion | Result |
|---|---|
| Blend uniformity | 94.3-103.2 |
| (range, %) | |
| Blend uniformity | 98.9 ± 3.0 |
| (Average ± SD, %) | |
| Hardness (kp) | 4.2 |
| Thickness (mm) | 3.33 |
| Disintegration time | 128 s |
| Weight variation range | +5% |

### Dissolution Testing of Veterinary Oral Solid Dosage Forms

### Dissolution Testing of Veterinary Tablets

Dissolution testing of the oral solid dosage forms can be carried out by many methods known to those of skill in the art. A variety of standard types of apparatus can be used, as well as different aqueous solutions that simulate to varying degrees the conditions likely to be encountered in the gastrointestinal tract. Table 40 presents suitable chromatographic conditions for the detection of bexagliflozin in fluid aspirated from a dissolution testing chamber, and Table 41 presents an example of veterinary tablet testing conditions that can be used to establish the rate of release of bexagliflozin into the test medium.

TABLE 40
Tablet Dissolution Testing
Chromatographic Conditions

| Parameter | Value | |
|---|---|---|
| Column | Agilent Zorbax SB-CN, | |
| | 75 mm × 4.6 mm, 3.5 μm | |
| Separation Type | Isocratic | |
| Mobile phase | 0.1% Phosphoric acid pH | |
| | 3.0 ± 0.1/Acetonitrile, (65/35) | |
| Flow Rate | 1.00 | mL/min |
| Injection Volume | 50 | μL |
| Column Temperature | 40° | C. |
| Detection Wavelength | 225 | nm |
| Run Time | 5 | minutes |
| Approx. Retention Time | 2 | minutes |

TABLE 41
Tablet Dissolution Conditions

| Parameter | Value |
|---|---|
| Lot Number | F6 - 201028 - 7 kp |
| Dissolution Media | 0.1N HCl |
| Volume | 500 mL |
| Apparatus | Type 2 (Paddles) |
| Sinker | None |
| Paddle Speed | 75 rpm (250 rpm after 60 minutes) |
| Number of Vessels | 6 |
| Time (minutes) | % Release |
| 5 | 57.3 |
| 10 | 73.8 |
| 15 | 81.8 |
| 20 | 86.8 |
| 25 | 88.2 |
| 30 | 88.9 |
| 45 | 89.0 |
| 60 | 89.1 |
| 75 | 93.5 |
| (Infinity)^{a} | |

^{a}After 15 min of stirring at 250 rpm

Table 42 shows the compositions of various experimental batches of bexagliflozin veterinary tablets, 15 mg, used for dissolution testing. Each batch contained 15 mg of bexagliflozin and each tablet weighed 375 mg. The amount of lactose monohydrate varied between 110.0 mg (formulation F5) and 161.0 mg (formulations F1, F3, F7, F9). The amount of microcrystalline cellulose varied between 117.0 mg (F13) and 185.4 (F11), whereas the amount of pregelatinized starch varied between 2.4 mg (F12) and 54 mg (F13), the amount of colloidal silicon dioxide between 0.6 mg (F12) and 13.5 mg (F13), the amount of magnesium stearate between 3.0 mg (F1, F6, F7-F11, F13) and 22.5 mg (F14), and the amount of palatant between 30 mg (F9, F10) and 44.2 mg (F3).

TABLE 42
Compositions of Experimental Bexagliflozin Veterinary Tablets, 15 mg

| | | Formulation (mg/tablet) | | | | | | | | | | | | | | | |
|---|---|---|---|---|---|---|---|---|---|---|---|---|---|---|---|---|---|
| Batch | F6 ª | F1 | F2 | F3 | F4 | F5 | F7 | F8 | F9 | F10 | F11 | F12 | F13 | F14 | F15 | F16 | F17 |
| Bex | 15.0 | 15.0 | 15.0 | 15.0 | 15.0 | 15.0 | 15.0 | 15.0 | 15.0 | 15.0 | 15.0 | 15.0 | 15.0 | 15.0 | 15.0 | 15.0 | 15.0 |
| Lac | 135.0 | 161.0 | 160.5 | 161.0 | 135.0 | 110.0 | 161.0 | 110.0 | 161.0 | 113.0 | 135.0 | 142.5 | 135.0 | 135.0 | 135.0 | 135.0 | 135.0 |
| MCC | 154.5 | 133.3 | 133.3 | 126.1 | 154.5 | 183.8 | 124.0 | 175.0 | 136.0 | 184.0 | 185.4 | 144.0 | 117.0 | 154.5 | 154.5 | 154.5 | 154.5 |
| PGS | 24.0 | 19.2 | 19.2 | 19.2 | 19.2 | 19.2 | 28.5 | 28.5 | 24.0 | 24.0 | 24.0 | 2.4 | 54.0 | 4.5 | 12.0 | 8.25 | 10.125 |
| SiO2 | 6.0 | 6.0 | 6.0 | 6.0 | 6.0 | 6.0 | 6.0 | 6.0 | 6.0 | 6.0 | 6.0 | 0.6 | 13.5 | 6.0 | 6.0 | 6.0 | 6.0 |
| MgSt | 3.0 | 3.0 | 3.5 | 3.5 | 3.5 | 3.5 | 3.0 | 3.0 | 3.0 | 3.0 | 3.0 | 33 | 3.0 | 22.5 | 15.0 | 18.75 | 16.875 |
| Pal | 37.5 | 37.5 | 37.5 | 44.2 | 41.8 | 37.5 | 37.5 | 37.5 | 30 | 30 | 37.5 | 37.5 | 37.5 | 37.5 | 37.5 | 37.5 | 37.5 |

| | |
|---|---|
| Sum | 375.0 |

^{a} Reference sample
Bex: bexagliflozin,
Lac: lactose monohydrate,
MCC: microcrystalline cellulose,
PGS: pregelatinized starch,
SiO2: colloidal silicon dioxide,
MgSt: magnesium stearate,
Pal: palatant

Three of the formulations were found to be dissimilar to the reference formulation (F6) by the criterion of f₂<50: F14, F16 and F17. In addition F15 was borderline (f₂=50). Equivalent dissolution by the f₂ criterion could be realized by tablets containing from 110.0 mg to 161.0 mg lactose monohydrate, 117.0 to 185.4 mg microcrystalline cellulose, from 19.2 to 54.0 mg pregelatinized starch, from to mg colloidal silicon dioxide, from 3.0 to 3.5 mg magnesium stearate and from 30 to 44.2 mg palatant (Table 42 and Table 43).

TABLE 43
Dissolution Results for Experimental Bexagliflozin Veterinary Tablets 15 mg

| | | Dissolution: 37° C./0.1N HCl/500 mL/75 rpm/Paddle (Tablet Hardness: 7 kp) | | | | | | | | | | | | | | | |
|---|---|---|---|---|---|---|---|---|---|---|---|---|---|---|---|---|---|
| | | | F3 | F4 | F5 | F6 ª | F7 | F8 | F9 | F10 | F11 | F12 ^{b} | F13 | F14 | F15 | F16 | F17 |
| Time | F1 | F2 | n = 6 | | | | | | | | | | | | | | |
| (min) | n = 12 | n = 12 | % Drug Release | | | | | | | | | | | | | | |
| 5 | 52.9 | 59.4 | 56.2 | 60.2 | 61.4 | 57.3 | 59.4 | 57.2 | 60.3 | 62.2 | 59.1 | 1.3 | 60.1 | 5.0 | 41.2 | 15.8 | 30.8 |
| 10 | 71.4 | 77.2 | 74.8 | 77.0 | 76.8 | 73.8 | 75.9 | 72.1 | 78.4 | 78.0 | 75.1 | 2.3 | 78.6 | 12.3 | 65.4 | 43.0 | 57.7 |
| 15 | 80.4 | 86.2 | 83.4 | 84.7 | 86.1 | 81.8 | 83.9 | 80.6 | 85.4 | 85.6 | 83.2 | 4.5 | 87.6 | 21.9 | 80.1 | 64.0 | 73.3 |
| 20 | N/A | N/A | N/A | N/A | N/A | 86.8 | 87.9 | 84.1 | 89.8 | 88.0 | 87.9 | 12.6 | 92.8 | 36.4 | 87.5 | 75.6 | 82.9 |
| 25 | N/A | N/A | N/A | N/A | N/A | 88.2 | 90.1 | 86.5 | 92.1 | 90.4 | 90.2 | 23.7 | 93.9 | 50.4 | 92.2 | 84.5 | 89.7 |
| 30 | 89.5 | 94.6 | 92.4 | 92.1 | 95.2 | 88.9 | 92.0 | 87.3 | 93.7 | 91.2 | 91.1 | 40.0 | 94.6 | 60.6 | 96.0 | 90.1 | 93.0 |
| 45 | 90.8 | 96.1 | 95.4 | 94.3 | 96.7 | 89.0 | 93.1 | 87.7 | 94.0 | 90.9 | 92.5 | 66.9 | 95.8 | 80.5 | 99.2 | 98.2 | 97.6 |
| 60 | 91.5 | 96.4 | 95.9 | 94.1 | 97.1 | 89.1 | 92.8 | 186.0 | 94.7 | 91.5 | 91.5 | 78.1 | 95.3 | 89.9 | 99.9 | 99.7 | 99.3 |
| final | 92.8 | 96.4 | 96.5 | 93.4 | 99.1 | 93.5 | 95.2 | 94.2 | 95.7 | 95.8 | 93.6 | 85.2 | 97.4 | 99.2 | 100.2 | 101.8 | 100.0 |
| f₂ ^{c} | 78 | 69 | 82 | 75 | 66 | (100) | 79 | 89 | 69 | 70 | 85 | 8 | 65 | 14 | 50 | 28 | 39 |

^{a} Reference sample
^{b} Could not be compressed to 7 kp (5 kp instead)
^{c} f₂ factor calculated from dissolution at 5, 10, 15, 30 min

1. A tablet formulation comprising bexagliflozin for administration to a companion animal, wherein in an in vitro dissolution test said formulation releases at least 85% of its bexagliflozin after 30 minutes in a solution of 0.1N HCl at 37±0.5° C. in a USP Apparatus 2 (a paddle apparatus) with a paddle speed of about 75 rpm.

2. The tablet formulation of claim 1, further comprising a palatant.

3. The tablet formulation of claim 2, wherein the palatant comprises tuna, salmon, cream, beef, peanut, catnip, chicken liver powder, poultry extract, avian hydrolyzed liver, butter, or bacon flavoring.

4.-6. (canceled)

7. The tablet formulation of claim 1, further comprising one or more fillers, one or more glidants, one or more lubricants, and one or more binders.

8. The tablet formulation of claim 1, comprising about 4 to 20% by weight of bexagliflozin.

9.-11. (canceled)

12. The tablet formulation of claim 2, comprising about 2 to 25% by weight of palatant.

13.-15. (canceled)

16. The tablet formulation of claim 7, comprising about 20 to 60% by weight of one or more fillers.

17.-20. (canceled)

21. The tablet formulation of claim 7, wherein said one or more fillers is microcrystalline cellulose.

22. The tablet formulation of claim 7, comprising about 0.5 to about 8% by weight of one or more glidants.

23.-25. (canceled)

26. The tablet formulation of claim 22, wherein said one or more glidants is colloidal silicon dioxide.

27. The tablet formulation of claim 7, comprising about 0.1 to 4% by weight of a lubricant.

28.-29. (canceled)

30. The tablet formulation of claim 27, wherein said one or more lubricants is magnesium stearate.

31. The tablet formulation of claim 7, comprising about 30 to about 50% by weight of one or more binders.

32.-34. (canceled)

35. The tablet formulation of claim 31, wherein the ratio of a first binder to a second binder is about 5.5:1.

36. The tablet formulation of claim 31, wherein said one or more binders is lactose monohydrate and pregelatinized starch.

37. The tablet formulation of claim 7, wherein said tablet comprises bexagliflozin, 15 mg; lactose monohydrate, between 110.0-160 mg; microcrystalline cellulose, between 117.0-185.4 mg; palatant, between 30-44.2 mg, pregelatinized starch, between 19.2-54 mg, colloidal silicon dioxide, between 6-13.5 mg; and magnesium stearate, between 3.0-3.5 mg.

38. The tablet formulation of claim 1, wherein the companion animal is a cat.

39. (canceled)

40. The tablet formulation of claim 1, wherein the formulation can be delivered daily with fewer than 1 dosing rejection per 30 dosing events.

41.-43. (canceled)

44. The formulation of claim 1, wherein after delivery to an appropriately constituted cohort of healthy adult cats in the fasted state, the formulation provides a mean plasma bexagliflozin Cₘₐₓ greater than 300 ng mL⁻¹ per mg kg⁻¹ when tested at a bexagliflozin dose level of 3 mg kg⁻¹ of body weight.

45. The formulation of claim 1, wherein after delivery to an appropriately constituted cohort of healthy adult cats in the fasted state, the formulation releases bexagliflozin in vivo to provide a Tₘₐₓ in a fasted cat that is between 0.1 and 2 hours.

46. The formulation of claim 1, wherein the tablet has a hardness between 5 to 10 kp and/or a friability of ≤1% by weight.

47. A tablet formulation of claim 1, comprising 15 mg of bexagliflozin and producing an f₂ value ≥50 when compared to a reference tablet of the formulation of Table 35, when tested by dissolution in USP Apparatus 2 (paddles) containing 500 mL of 0.1 N HCl at 37° C.±0.5° C., stirred at 75 rpm.

48. A tablet formulation of claim 1, comprising the following composition per tablet: bexagliflozin, between 10-20 mg; lactose monohydrate, between 17.5-27.5 mg; microcrystalline cellulose, between 20.5-30.5 mg; palatant, between 7.5-11 mg, pregelatinized starch, between 3-5 mg, colloidal silicon dioxide, between 1.5-2.5 mg; and magnesium stearate, between 0.75-1.25 mg.

49. A tablet formulation of claim 1, comprising 15 mg of bexagliflozin and producing an f₂ value ≥50 when compared to a reference tablet of the formulation of Table 38, when tested by dissolution in USP Apparatus 2 (paddles) containing 500 mL of 0.1 N HCl at 37° C.±0.5° C., stirred at 75 rpm.

50. A method for treating an animal suffering from a disease or syndrome susceptible to treatment with an SGLT2 inhibitor, comprising a step of administering to the animal the formulation of claim 1.

51. A liquid formulation containing bexagliflozin, 30 mg/mL, ethanol, 125 to 175 mg/mL, glycerol, 250 to 300 mg/mL, PEG-400, 125 to 175 mg/mL, polysorbate 80, 25 to 75 mg/mL, one or more optional palatants, 1 to 15 mg/mL and with the remainder comprising an aqueous buffer with pH between 6.0 and 8.0.

52. A batch of bexagliflozin veterinary tablets that include 15 mg of bexagliflozin per tablet wherein, upon administration to an appropriately constituted cohort of healthy fasted subjects, a first representative sample set of tablets from the batch provides on one occasion a first mean logarithm of the Cₘₐₓ and a first mean logarithm of the AUC₀₋ₜ, and a second representative sample of tablets from the batch produces on a different occasion a second mean logarithm of the Cₘₐₓ and a second mean logarithm of the AUC₀₋ₜ, and wherein the differences between the first and second mean logarithms of the Cₘₐₓ and between the first and second mean logarithms of the AUC₀₋ₜ both exhibit 90% confidence intervals, the endpoints of which lie between −0.51083 and +0.51083.

53.-54. (canceled)

55. A batch of bexagliflozin veterinary tablets that include 15 mg of bexagliflozin per tablet wherein, upon administration to an appropriately constituted cohort of healthy fasted subjects, a representative sample set of tablets from the batch provides a first mean logarithm of the Cₘₐₓ and a first mean logarithm of the AUC₀₋ₜ, and a representative sample of tablets from a reference batch of 15 mg bexagliflozin veterinary tablets produces a second mean logarithm of the Cₘₐₓ and a second mean logarithm of the AUC₀₋ₜ, and wherein the differences between the first and second mean logarithms of the Cₘₐₓ and between the first and second mean logarithms of the AUC₀₋ₜ both exhibit 90% confidence intervals, the endpoints of which lie between −0.51083 and +0.51083.

56.-57. (canceled)
